(19) United States
(12) Patent Application Publication
(10) Pub. No.: US 2025/0009714 A1
(43) Pub. Date: Jan. 9, 2025

(54) SELECTION AND TREATMENT OF SUBJECTS HAVING A CIRCULATING MYELOID CELL INFLAMMATORY PHENOTYPE

(71) Applicants: Tranquis Therapeutics, Inc., San Mateo, CA (US); The Board of Trustees of The Leland Stanford Junior University, Stanford, CA (US)

(72) Inventors: Sanjay Kumar Kakkar, Bormio (IT); Jonas O'Gara Hannestad, Baltimore, MD (US); Hope Lancero, Redwood City, CA (US); Khoa Dinh Nguyen, Stanford, CA (US); Edgar G. Engleman, Stanford, CA (US)

(21) Appl. No.: 18/707,062

(22) PCT Filed: Nov. 1, 2022
(86) PCT No.: PCT/US2022/079079

§ 371 Date: May 2, 2024

Related U.S. Application Data
(60) Provisional application No. 63/274,931, filed on Nov. 2, 2021.

(51) Int. Cl. A61K 31/4184; A61P 25/28; A61P 29/00; G01N 33/50; G01N 33/68

(52) U.S. Cl. CPC A61K 31/4184; A61P 25/28; A61P 29/00; G01N 33/5091; G01N 33/6896; G01N 2333/4703; G01N 2333/4709; G01N 2800/2814; G01N 2800/52

(57) ABSTRACT

Methods are presented for selecting a subject for treatment with a 2-arylbenzimidazole compound of Formula I, or salt, hydrate, deuterated analog, or fluorinated analog thereof, based on the inflammatory phenotype of circulating myeloid cells. Methods are also provided for treating, slowing the progression of, and delaying the onset of neurodegenerative disease in a subject who has not been diagnosed with a neurodegenerative disease but is at risk of developing a neurodegenerative disease or cognitive impairment, comprising selecting the subject for treatment based on the inflammatory phenotype of circulating myeloid cells and administering a therapeutically effective amount of compound of Formula I or salt, hydrate, deuterated analog, or fluorinated analog thereof.

### 1. CROSS-REFERENCE TO RELATED APPLICATIONS

This application claims the benefit of and priority to U.S. Provisional Application No. 63/274,931, filed Nov. 2, 2021, which is hereby incorporated by reference in its entirety.

### 2. BACKGROUND OF THE INVENTION

TQS-168 (2-(4-tert-butylphenyl)-1H-benzimidazole), previously known as ZLN-005, is known to be an activator of Ppargc1α (PGC-1α) expression (Zhang et al., Diabetes 62:1297-1307 (2013)), although its immediate target—its direct molecular binding partner—is unknown. TQS-168 has previously been shown to suppress myeloid-mediated inflammation of the central nervous system, and to reduce disease severity in murine models of neurodegenerative diseases in which neuroinflammation contributes to the underlying pathophysiology, including Parkinson's disease, Alzheimer's disease, and amyotrophic lateral sclerosis (ALS) (U.S. Pat. Nos. 10,272,070; 10,583,125; and 10,653,669, the disclosures of which are incorporated herein by reference in their entireties). TQS-168 has also been shown to suppress metabolic dysfunction in microglia in older mice, inhibit inflammatory cytokine production in microglia in older mice, suppress systemic inflammation in older mice, and alleviate behavioral dysfunction in older mice (U.S. Pat. No. 10,653,669, the disclosure of which is incorporated herein by reference in its entirety).

Myeloid-mediated inflammation of the CNS, and in particular the inflammatory state of microglia in the central nervous system, cannot readily be assessed in vivo. There is, therefore, a need for a clinically accessible marker that can be used to identify and select patients for treatment with TQS-168 and other 2-aryl-benzimidazole compounds, and that can be used to monitor and to titrate treatment of such patients. There is a particular need for a method to assess and monitor the efficacy of activators of Ppargc1α (PGC-1α) expression in human ALS patients.

### 3. SUMMARY OF THE INVENTION

As detailed in the experimental examples in this disclosure, we have discovered that the compound of Formula I suppresses, and or induces/promotes the suppression of pro-inflammatory phenotype of circulating myeloid cells from ALS patients in vitro. As shown herein, individuals with ALS display a greater expression of pro-inflammatory CD14+CD16+ circulating myeloid cells as compared to healthy populations. Presentation of Compound I to a biological sample from ALS patients showed a suppression and reduction in CD14+CD16+ circulating myeloid cells in vitro. Suppression of the pro-inflammatory CD14+CD16+ circulating myeloid cells will be effective in treating, preventing the onset of, and or slowing the progression of neuroinflammation and neurodegenerative diseases expressing proinflammatory phenotype of circulating myeloid cells.

Accordingly, in a first aspect, methods are provided for selecting a subject for treatment with a compound of Formula I.

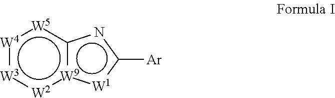

or a salt, hydrate, deuterated analog, or fluorinated analog thereof, wherein
   Ar is:

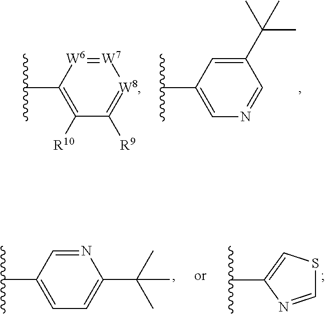

W¹ is chosen from N—R¹, O, and S, or when W⁹ is N, W¹ may additionally be C—R⁵⁰;
W² is C—R² or N;
W³ is C—R³ or N;
W⁴ is C—R⁴ or N;
W⁵ is C—R⁵ or N;
W⁶ is C—R⁶ or N;
W⁷ is C—R⁷ or N;
W⁸ is C—R⁸ or N;
W⁹ is C, or when W¹ is C—R⁵⁰, W⁹ may be N;
R¹ is selected from H, (C₁-C₃)alkyl, —CH₂OC(═O)R³⁰, —CH₂OP(═O)OR⁴⁰OR⁴¹, —C(═O)OR⁴², and —C(═O)R⁴³;
R², R³, R⁴, and R⁵ are selected independently from hydrogen, deuterium, halogen, perfluoro(C₁-C₄)alkyl, (C₁-C₄)alkyl, (C₁-C₄)alkoxy, perfluoro(C₁-C₄)alkoxy, (C₁-C₄)acyl, (C₁-C₄)alkoxy(C₁-C₄)alkyl, hydroxy(C₁-C₄)alkyl, hydroxy, carboxy, (C₁-C₄)alkoxycarbonylamino, carboxamido, (C₁-C₄)alkylaminocarbonyl, cyano, acetoxy, nitro, amino, (C₁-C₄)alkylamino, di(C₁-C₄)alkylamino, mercapto, (C₁-C₄)alkylthio, aminosulfonyl, (C₁-C₄)alkylsulfonyl, and (C₁-C₄)acylamino;
R⁶ and R¹⁰ are selected independently from hydrogen, deuterium, halo, (C₁-C₃)alkyl, perfluoro(C₁-C₃)alkyl, hydroxy, (C₁-C₃)alkoxy, perfluoro(C₁-C₃)alkoxy, and amino;
R⁷ and R⁹ are selected independently from hydrogen, deuterium, hydroxy, cyano, amino, halogen, halo(C₁-C₄)alkyl, (C₁-C₄)alkoxy, halo(C₁-C₄)alkoxy,

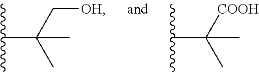

R⁸ is selected from hydrogen, deuterium, halogen, halo(C₁-C₄)alkyl, (C₁-C₄)alkyl, (C₁-C₄)alkoxy, halo(C₁-C₄)alkoxy, cyano, phenyl, phenoxy, benzyloxy, amino,

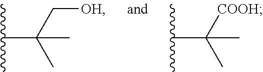

R³⁰ is selected from (C₁-C₁₀)hydrocarbyl, (C₁-C₁₀)hydrocarbyl substituted with amino, (C₁-C₁₀)hydrocarbyl substituted with (C₁-C₄)hydrocarbyl, (C₁-C₁₀)hydrocarbyl substituted with carboxyl, carboxy, (C₁-C₆)alkoxycarbonyl, (C₁-C₆)alkoxycarbonylamino, methylthio, heterocyclyl, (C₁-C₁₀)oxaalkyl, CHR⁴⁴NHR⁴⁵ and guanidine;
R⁴⁰ and R⁴¹ are selected independently from hydrogen (C₁-C₆)hydrocarbyl;
R⁴² is (C₁-C₅)alkyl;
R⁴³ is (C₁-C₃)alkyl,
R⁴⁴ is selected from any naturally occurring amino acid sidechain;
R⁴⁵ is selected from H, methyl, and (C₁-C₄)alkoxycarbonyl; and
R⁵⁰ is H or (C₁-C₃)alkyl.
   The method comprises measuring the baseline concentration of CD14+CD16+ myeloid cells in a biological sample containing circulating myeloid cells from the subject, comparing the baseline concentration of circulating CD14+CD16+ myeloid cells to a predetermined threshold concentration, and if the patient baseline concentration of CD14+CD16+ myeloid cells is greater than the predetermined threshold, thereby indicating a circulating myeloid cell inflammatory phenotype, selecting the patient for treatment.

In a further aspect, methods are provided for deselecting the subject for treatment if the baseline concentration of circulating CD14+CD16+ myeloid cells is less than the predetermined threshold. A lower baseline concentration of circulating CD14+CD16+ myeloid cells compared to the predetermined threshold indicates a circulating myeloid cell noninflammatory phenotype.

In some embodiments of the methods, the predetermined threshold is the concentration of circulating CD14+CD16+ myeloid cells in a biological sample containing circulating myeloid cells from a healthy individual.

In some embodiments of the methods, the patient has amyotrophic lateral sclerosis (ALS).

In a further aspect, methods are provided administering to the subject who is selected for treatment a therapeutically effective amount of a compound of Formula I or a salt, hydrate, deuterated analog, or fluorinated analog thereof.

In some embodiments of the methods, the therapeutically effective amount is an amount sufficient to suppress the inflammatory phenotype of circulating myeloid cells.

In some embodiments of the methods, the subject selected for treatment with the compound of Formula I or a salt, hydrate, deuterated analog, or fluorinated analog thereof has not previously been diagnosed with, but is at risk for developing neuroinflammation.

In some embodiments of the methods, the subject selected for treatment with the compound of Formula I or a salt, hydrate, deuterated analog, or fluorinated analog thereof has not previously been diagnosed with, but is at risk for developing, a neurodegenerative disease.

In some embodiments of the methods, the subject has at least one ApoE4 allele.

In some embodiments of the methods, wherein the subject has family history of neurodegenerative disease.

In some embodiments of the methods, the subject has a mild cognitive impairment.

In some embodiments of the methods, the subject has TREM2 heterozygous or homozygous mutations.

In some embodiments of the methods, the subject has positive amyloid-O (Aβ) or tau protein in cerebrospinal fluid (CSF).

In some embodiments of the methods, the subject has had a positive amyloid-O (Aβ) PET scan.

In some embodiments of the methods, the subject has GRN heterozygous or homozygous mutations. In some embodiments of the methods, the subject has reduced progranulin levels.

In some embodiments of the methods, the subject selected for treatment with the compound of Formula I or a salt, hydrate, deuterated analog, or fluorinated analog thereof, has not previously been diagnosed with cognitive impairment, but is at least 40 years old. In some embodiments, the subject is at least 45 years old. In some embodiments, the subject is at least 50 years old. In some embodiments, the subject is at least 55 years old. In some embodiments, the subject is at least 60 years old. In some embodiments, the subject is at least 65 years old. In some embodiments, the subject is at least 70 years old. In some embodiments, the subject is at least 75 years old. In some embodiments, the subject is at least 80 years old. In some embodiments, the subject is at least 85 years old.

In another aspect, methods are provided for treating a subject who has not previously been diagnosed with, but is at risk for developing, neuroinflammation and/or a neurodegenerative disease. The methods comprise:
a) selecting a patient for treatment, according to the methods previously mentioned; and
b) administering to the subject who is selected for treatment at least a first dose of a compound of Formula I

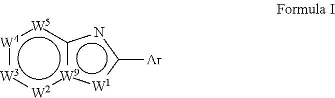

or a salt, hydrate, deuterated analog, or fluorinated analog thereof, wherein
Ar is:

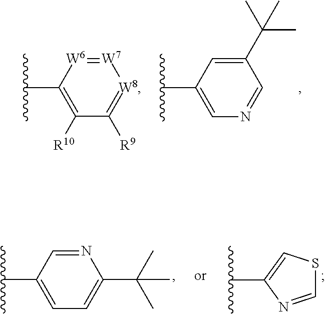

W¹ is chosen from N—R¹, O, and S, or when W⁹ is N, W¹ may additionally be C—R⁵⁰;
W² is C—R² or N;
W³ is C—R³ or N;
W⁴ is C—R⁴ or N;
W⁵ is C—R⁵ or N;
W⁶ is C—R⁶ or N;
W⁷ is C—R⁷ or N;
W⁸ is C—R⁸ or N;
W⁹ is C, or when W¹ is C—R⁵⁰, W⁹ may be N;
R¹ is selected from H, (C₁-C₃)alkyl, —CH₂OC(═O)R³⁰, —CH₂OP(═O)OR⁴⁰OR⁴¹, —C(═O)OR⁴², and —C(═O)R⁴³;
R², R³, R⁴, and R⁵ are selected independently from hydrogen, deuterium, halogen, perfluoro(C₁-C₄)alkyl, (C₁-C₄)alkyl, (C₁-C₄)alkoxy, perfluoro(C₁-C₄)alkoxy, (C₁-C₄)acyl, (C₁-C₄)alkoxy(C₁-C₄)alkyl, hydroxy(C₁-C₄)alkyl, hydroxy, carboxy, (C₁-C₄)alkoxycarbonylamino, carboxamido, (C₁-C₄)alkylaminocarbonyl, cyano, acetoxy, nitro, amino, (C₁-C₄)alkylamino, di(C₁-C₄)alkylamino, mercapto, (C₁-C₄)alkylthio, aminosulfonyl, (C₁-C₄)alkylsulfonyl, and (C₁-C₄)acylamino;
R⁶ and R¹⁰ are selected independently from hydrogen, deuterium, halo, (C₁-C₃)alkyl, perfluoro(C₁-C₃)alkyl, hydroxy, (C₁-C₃)alkoxy, perfluoro(C₁-C₃)alkoxy, and amino;
R⁷ and R⁹ are selected independently from hydrogen, deuterium, hydroxy, cyano, amino, halogen, halo(C₁-C₄)alkyl, (C₁-C₄)alkoxy, halo(C₁-C₄)alkoxy,

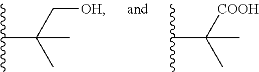

R⁸ is selected from hydrogen, deuterium, halogen, halo(C₁-C₄)alkyl, (C₁-C₄)alkyl, (C₁-C₄)alkoxy, halo(C₁-C₄)alkoxy, cyano, phenyl, phenoxy, benzyloxy, amino,

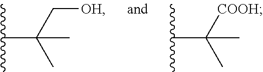

R³⁰ is selected from (C₁-C₁₀)hydrocarbyl, (C₁-C₁₀)hydrocarbyl substituted with amino, (C₁-C₁₀)hydrocarbyl substituted with (C₁-C₄)hydrocarbyl, (C₁-C₁₀)hydrocarbyl substituted with carboxyl, carboxy, (C₁-C₆)alkoxycarbonyl, (C₁-C₆)alkoxycarbonylamino, methylthio, heterocyclyl, (C₁-C₁₀)oxaalkyl, CHR⁴⁴NHR⁴⁵ and guanidine;
R⁴⁰ and R⁴¹ are selected independently from hydrogen (C₁-C₆)hydrocarbyl;
R⁴² is (C₁-C₅)alkyl;
R⁴³ is (C₁-C₃)alkyl,
R⁴⁴ is selected from any naturally occurring amino acid sidechain;
R⁴⁵ is selected from H, methyl, and (C₁-C₄)alkoxycarbonyl; and
R⁵⁰ is H or (C₁-C₃)alkyl.

In some embodiments, additional methods are provided for treating a subject who has not previously been diagnosed with, but is at risk for developing, neuroinflammation and/or a neurodegenerative disease. The method further comprises:
c) determining the post-dose concentration of circulating CD14+CD16+ myeloid cells in a biological sample obtained from the subject;
d) comparing the subject's post-dose concentration to the subject's baseline concentration; and
e) if the post-dose concentration is less than the baseline concentration, administering a plurality of subsequent doses at the same or at higher amounts.

In some embodiments, additional methods are provided for treating a subject who has not previously been diagnosed with, but is at risk for developing, neuroinflammation and/or a neurodegenerative disease. The method further comprises:
f) if the post-dose concentration is not less than the baseline blood concentration, repeating steps (a)-(d) with successively increased doses of the compound of Formula I until either the subject's post-dose concentration of circulating CD14+CD16+ myeloid cells is lower than the subject's baseline concentration, or a predetermined maximum dose of the compound is reached.

In some embodiments, additional methods are provided for treating a subject who has not previously been diagnosed with, but is at risk for developing, neuroinflammation and/or a neurodegenerative disease. The method further comprises:
g) deselecting the subject for further treatment if no post-dose concentration of CD14+CD16+ myeloid cells is lower than the patient's baseline concentration.

In another aspect, methods are provided for treating a subject who has not previously been diagnosed with cognitive impairment, but is at least 40 years old. The method comprises:
a) selecting a patient for treatment according to the method of claim 1; and
b) administering to the subject who is selected for treatment at least a first dose of a compound of Formula I

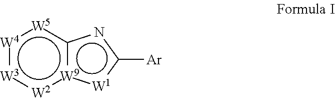

or a salt, solvate, hydrate, deuterated analog, or fluorinated analog thereof, wherein
Ar is:

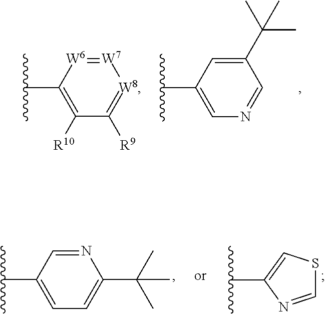

W¹ is chosen from N—R¹, O, and S, or when W⁹ is N, W¹ may additionally be C—R⁵⁰;
W² is C—R² or N;
W³ is C—R³ or N;
W⁴ is C—R⁴ or N;
W⁵ is C—R⁵ or N;
W⁶ is C—R⁶ or N;
W⁷ is C—R⁷ or N;
W⁸ is C—R⁸ or N;
W⁹ is C, or when W¹ is C—R⁵⁰, W⁹ may be N;
R¹ is selected from H, (C₁-C₃)alkyl, —CH₂OC(═O)R³⁰, —CH₂OP(═O)OR⁴⁰OR⁴¹, —C(═O)OR⁴², and —C(═O)R⁴³;
R², R³, R⁴, and R⁵ are selected independently from hydrogen, deuterium, halogen, perfluoro(C₁-C₄)alkyl, (C₁-C₄)alkyl, (C₁-C₄)alkoxy, perfluoro(C₁-C₄)alkoxy, (C₁-C₄)acyl, (C₁-C₄)alkoxy(C₁-C₄)alkyl, hydroxy(C₁-C₄)alkyl, hydroxy, carboxy, (C₁-C₄)alkoxycarbonylamino, carboxamido, (C₁-C₄)alkylaminocarbonyl, cyano, acetoxy, nitro, amino, (C₁-C₄)alkylamino, di(C₁-C₄)alkylamino, mercapto, (C₁-C₄)alkylthio, aminosulfonyl, (C₁-C₄)alkylsulfonyl, and (C₁-C₄)acylamino;
R⁶ and R¹⁰ are selected independently from hydrogen, deuterium, halo, (C₁-C₃)alkyl, perfluoro(C₁-C₃)alkyl, hydroxy, (C₁-C₃)alkoxy, perfluoro(C₁-C₃)alkoxy, and amino;
R⁷ and R⁹ are selected independently from hydrogen, deuterium, hydroxy, cyano, amino, halogen, halo(C₁-C₄)alkyl, (C₁-C₄)alkoxy, halo(C₁-C₄)alkoxy,

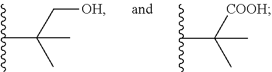

R⁸ is selected from hydrogen, deuterium, halogen, halo(C₁-C₄)alkyl, (C₁-C₄)alkyl, (C₁-C₄)alkoxy, halo(C₁-C₄)alkoxy, cyano, phenyl, phenoxy, benzyloxy, amino,

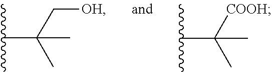

R³⁰ is selected from (C₁-C₁₀)hydrocarbyl, (C₁-C₁₀)hydrocarbyl substituted with amino, (C₁-C₁₀)hydrocarbyl substituted with (C₁-C₄)hydrocarbyl, (C₁-C₁₀)hydrocarbyl substituted with carboxyl, carboxy, (C₁-C₆)alkoxycarbonyl, (C₁-C₆)alkoxycarbonylamino, methylthio, heterocyclyl, (C₁-C₁₀)oxaalkyl, CHR⁴⁴NHR⁴⁵ and guanidine;
R⁴⁰ and R⁴¹ are selected independently from hydrogen (C₁-C₆)hydrocarbyl;
R⁴² is (C₁-C₅)alkyl;
R⁴³ is (C₁-C₃)alkyl,
R⁴ is selected from any naturally occurring amino acid sidechain;
R⁴⁵ is selected from H, methyl, and (C₁-C₄)alkoxycarbonyl; and
R⁵⁰ is H or (C₁-C₃)alkyl.

In some embodiments, additional methods are provided for treating a subject who has not previously been diagnosed with cognitive impairment, but is at least 40 years old. The method comprises:
c) determining the post-dose concentration of circulating CD14+CD16+ myeloid cells in a biological sample obtained from the subject;
d) comparing the subject's post-dose concentration to the subject's baseline concentration; and
e) if the post-dose concentration is less than the baseline concentration, administering a plurality of subsequent doses at the same or at higher amounts.

In some embodiments, additional methods are provided for treating a subject who has not previously been diagnosed with cognitive impairment, but is at least 40 years old. The method comprises:
f) if the post-dose concentration is not less than the baseline blood concentration, repeating steps (a)-(d) with successively increased doses of the compound of Formula I until either the subject's post-dose concentration of circulating CD14+CD16+ myeloid cells is lower than the subject's baseline concentration, or a predetermined maximum dose of the compound is reached.

In some embodiments, additional methods are provided for treating a subject who has not previously been diagnosed with cognitive impairment, but is at least 40 years old. The method comprises:
g) deselecting the subject for further treatment if no post-dose concentration of CD14+CD16+ myeloid cells is lower than the patient's baseline concentration.

In another aspect, methods are provided for treating a subject who has an inflammatory phenotype of circulating myeloid cells. The method comprises:
administering to the subject a therapeutically effective amount of a compound of Formula I

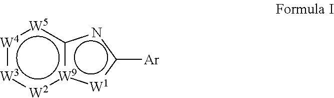

or a salt, solvate, hydrate, deuterated analog, or fluorinated analog thereof, wherein
Ar is:

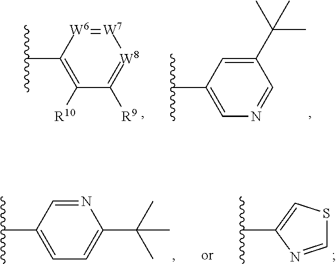

W¹ is chosen from N—R¹, O, and S, or when W⁹ is N, W¹ may additionally be C—R⁵⁰;
W² is C—R² or N;
W³ is C—R³ or N;
W⁴ is C—R⁴ or N;
W⁵ is C—R⁵ or N;
W⁶ is C—R⁶ or N;
W⁷ is C—R⁷ or N;
W⁸ is C—R⁸ or N;
W⁹ is C, or when W¹ is C—R⁵⁰, W⁹ may be N;
R¹ is selected from H, (C₁-C₃)alkyl, —CH₂OC(═O)R³⁰, —CH₂OP(═O)OR⁴⁰OR⁴¹, —C(═O)OR⁴², and —C(═O)R⁴³;
R², R³, R⁴, and R⁵ are selected independently from hydrogen, deuterium, halogen, perfluoro(C₁-C₄)alkyl, (C₁-C₄)alkyl, (C₁-C₄)alkoxy, perfluoro(C₁-C₄)alkoxy, (C₁-C₄)acyl, (C₁-C₄)alkoxy(C₁-C₄)alkyl, hydroxy(C₁-C₄)alkyl, hydroxy, carboxy, (C₁—C₄)alkoxycarbonylamino, carboxamido, (C₁-C₄)alkylaminocarbonyl, cyano, acetoxy, nitro, amino, (C₁-C₄)alkylamino, di(C₁-C₄)alkylamino, mercapto, (C₁-C₄)alkylthio, aminosulfonyl, (C₁-C₄)alkylsulfonyl, and (C₁-C₄)acylamino;
R⁶ and R¹⁰ are selected independently from hydrogen, deuterium, halo, (C₁-C₃)alkyl, perfluoro(C₁-C₃)alkyl, hydroxy, (C₁-C₃)alkoxy, perfluoro(C₁-C₃)alkoxy, and amino;
R⁷ and R⁹ are selected independently from hydrogen, deuterium, hydroxy, cyano, amino, halogen, halo(C₁-C₄)alkyl, (C₁-C₄)alkoxy, halo(C₁-C₄)alkoxy,

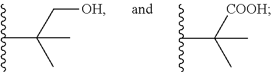

and R⁸ is selected from hydrogen, deuterium, halogen, halo(C₁-C₄)alkyl, (C₁-C₄)alkyl, (C₁-C₄)alkoxy, halo(C₁-C₄)alkoxy, cyano, phenyl, phenoxy, benzyloxy, amino,

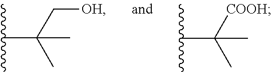

R³⁰ is selected from (C₁-C₁₀)hydrocarbyl, (C₁-C₁₀)hydrocarbyl substituted with amino, (C₁-C₁₀)hydrocarbyl substituted with (C₁-C₄)hydrocarbyl, (C₁-C₁₀)hydrocarbyl substituted with carboxyl, carboxy, (C₁-C₆)alkoxycarbonyl, (C₁-C₆)alkoxycarbonylamino, methylthio, heterocyclyl, (C₁-C₁₀)oxaalkyl, CHR⁴⁴NHR⁴⁵ and guanidine;
R⁴⁰ and R⁴¹ are selected independently from hydrogen (C₁-C₆)hydrocarbyl;
R⁴² is (C₁-C₅)alkyl;
R⁴³ is (C₁-C₃)alkyl,
R⁴ is selected from any naturally occurring amino acid sidechain;
R⁴⁵ is selected from H, methyl, and (C₁-C₄)alkoxycarbonyl; and
R⁵⁰ is H or (C₁-C₃)alkyl.

In some embodiments, additional methods are provided for treating a subject who has an inflammatory phenotype of circulating myeloid cells. The additional method comprises:
b) determining the post-dose concentration of circulating CD14+CD16+ myeloid cells in a biological sample obtained from the subject;
c) comparing the subject's post-dose concentration to the subject's baseline concentration; and
d) if the post-dose concentration is less than the baseline concentration, administering a plurality of subsequent doses at the same or at higher amounts.

In some embodiments, additional methods are provided for treating a subject who has an inflammatory phenotype of circulating myeloid cells. The additional method comprises:
e) if the post-dose concentration is not less than the baseline blood concentration, repeating steps (a)-(c) with successively increased doses of the compound of Formula I until either the subject's post-dose concentration of circulating CD14+CD16+ myeloid cells is lower than the subject's baseline concentration, or a predetermined maximum dose of the compound is reached.

In some embodiments, additional methods are provided for treating a subject who has an inflammatory phenotype of circulating myeloid cells. The additional method comprises:
g) deselecting the subject for further treatment if no post-dose concentration of CD14+CD16+ monocytes is lower than the patient's baseline concentration.

In some embodiments of the methods, the circulating CD14+CD16+ myeloid cells are monocytes.

In some embodiments of the methods, the compound is administered orally.

In some embodiments of the methods, the administered dose is about 200 mg to about 800 mg. In some embodiments of the methods, the administered dose is about 300 mg to about 700 mg. In some embodiments of the methods, the administered dose ranges is about 400 mg to about 600 mg.

In some embodiments of the methods, the dose is 400 mg, 450 mg, or 500 mg.

In some embodiments of the methods, the dose is administered daily. In some embodiments of the methods, the dose is administered once daily as a single undivided dose.

In some embodiments of the methods, the biological sample is whole blood.

In some embodiments of the methods, the concentration of CD14+CD16+ circulating myeloid cells is determined by measuring at least one of CD14 and CD16 polypeptide, CD14 and CD16 mRNA, and CD14 and CD16 cDNA in the biological sample. In some embodiments of the methods, the concentration of CD14+CD16+ monocytes is determined by measuring CD14 and CD16 polypeptide. In some embodiments of the methods, the concentration of CD14+CD16+ monocytes in the sample is measured by using one or more reagents that specifically bind CD14 or CD16 polypeptide. In some embodiments of the methods, the reagent is selected from the group consisting of an antibody, an antibody derivative, and an antigen-binding antibody fragment. In some embodiments of the methods, the concentration of CD14+CD16+ monocytes is determined by flow cytometry.

In some embodiments of the methods, the levels of CD14 and CD16 polypeptide are determined by detecting the amount of a transcribed polynucleotide or portion thereof. In some embodiments of the methods, the transcribed polynucleotide is an mRNA. In some embodiments of the methods, the transcribed polynucleotide is a cDNA. In some embodiments of the methods, the detecting further comprises amplifying the transcribed polynuclotide or portion thereof.

In some embodiments of the methods, the compound is selected from the compounds of Compound I

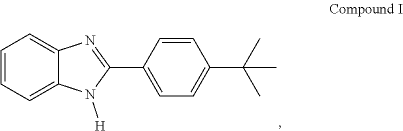 [SMILES: CC(C)(C)c1ccc(-c2nc3ccccc3[nH]2)cc1]

### and Compound II

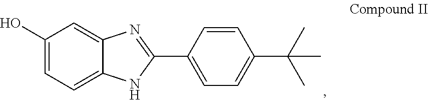 [SMILES: CC(C)(C)c1ccc(-c2nc3cc(O)ccc3[nH]2)cc1]

or a pharmaceutically acceptable salt, hydrate, or deuterated analog thereof.

In some embodiments of the methods, the compound is Compound I or a pharmaceutically acceptable salt, hydrate, or deuterated analog thereof.

In some embodiments of the methods, the compound is Compound II or a pharmaceutically acceptable salt, hydrate, or deuterated analog thereof.

In some embodiments of the methods, the compound or salt, hydrate, deuterated analog, or fluorinated analog thereof is administered in a suspension, a solution, or in a solid dosage form.

In some embodiments of the methods, the solid dosage form is a capsule. In some embodiments of the methods, the solid dosage form is a tablet.

### 4. BRIEF DESCRIPTION OF THE SEVERAL VIEWS OF THE DRAWINGS

These and other features, aspects, and advantages of the present invention will become better understood with regard to the following description, and accompanying drawings, where:

FIG. 1A shows the gating procedure employed in a FACS assay of mouse LPS-induced blood samples to detect and quantify Ly6C^{hi} cells. FIG. 1B shows that in vitro incubation of wild type mouse blood samples with LPS increases the percentage of Ly6C^{hi} cells, and that incubation with TQS-168 suppresses this increase in inflammatory myeloid cells. FIG. 1C illustrates the experimental design.

FIG. 2A plots plasma concentration of TQS-168 over time after a single oral dose of 50 mg/mL TQS-168 in mice. FIG. 2B is a timeline representative of the protocol of the SOD1 mouse study.

FIG. 3A is a bar graph showing that TQS-168 reduces the fraction of inflammatory monocytes in SOD1 mice. FIG. 3B is a bar graph showing that TQS-168 regulates plasma levels of anti and pro-inflammatory cytokines in SOD1 mice.

FIG. 4A compares percentage survival vs. time after 110 days in SOD1 mice treated with oral doses of 50 mg/kg TQS-168. FIG. 4B compares the survival age in days of two populations, SOD1 mice and SOD1 mice treated with TQS-168 50 mg/kg administered orally.

FIG. 5A is a FACS assay showing that the percentage of CD14+CD16+ monocytes in peripheral blood samples of ALS patients is higher than the percentage of CD14+CD16+ monocytes in peripheral blood samples of healthy normal subjects. FIG. 5B outlines the experimental protocol for assessing the effects of TQS-168 on the inflammatory phenotype of circulating myeloid cells in peripheral blood samples of ALS patients and control health donors, and FIG. 5C is a bar graph showing the results of a four-hour ex vivo incubation of the peripheral blood samples with increasing concentrations of TQS-168, demonstrating that TQS-168 dose-dependently reduces the percentage of CD14+CD16+ cells in the whole blood of ALS patients.

### 5. DETAILED DESCRIPTION OF THE INVENTION

### 5.1. Definitions

Unless defined otherwise, all technical and scientific terms used herein have the meaning commonly understood by a person skilled in the art to which this invention belongs.

An individual subject has an “inflammatory phenotype of circulating myeloid cells” if the concentration of CD14+CD16+ myeloid cells in a sample drawn from the subject's peripheral circulation exceeds a predetermined threshold concentration. An individual subject has a “non-inflammatory phenotype of circulating myeloid cells” if the concentration of CD14+CD16+ myeloid cells in a sample drawn from the subject's peripheral circulation does not exceed a predetermined threshold concentration, as further defined below. The phrase “suppression of the inflammatory phenotype of circulating myeloid cells” refers to causing the concentration of CD14+CD16+ cells in a sample drawn from the subject's peripheral circulation to drop below a predetermined threshold concentration.

The terms “individual,” “host,” and “subject” are used interchangeably, and refer to an animal to be treated, including but not limited to humans and non-human primates; rodents, including rats and mice; bovines; equines; ovines; felines; and canines. “Patient” refers to a human subject.

The terms “treating,” “treatment,” and grammatical variations thereof are used in the broadest sense understood in the clinical arts. Accordingly, the terms do not require cure or complete remission of disease and encompass obtaining any clinically desired pharmacologic and/or physiologic effect. Unless otherwise specified, “treating” and “treatment” do not encompass prophylaxis.

The phrase “therapeutically effective amount” refers to the amount of a compound that, when administered to a mammal or other subject for treating a disease, condition, or disorder, is sufficient to effect treatment of the disease, condition, or disorder. The “therapeutically effective amount” may vary depending on the compound, the disease and its severity and the age, weight, etc., of the subject to be treated.

The term “pharmaceutically acceptable salt” refers to a salt that is acceptable for administration to a subject. Examples of pharmaceutically acceptable salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, and undecanoate.

Other examples of pharmaceutically salts include anions of the compounds of the present disclosure compounded with a suitable cation such as N+, NH4+, and NW4+(where W can be a C₁-C₈ alkyl group), and the like. For therapeutic use, salts of the compounds of the present disclosure can be pharmaceutically acceptable. However, salts of acids and bases that are non-pharmaceutically acceptable may also find use, for example, in the preparation or purification of a pharmaceutically acceptable compound.

Compounds included in the present compositions and methods that are basic in nature are capable of forming a wide variety of salts with various inorganic and organic acids. The acids that can be used to prepare pharmaceutically acceptable acid addition salts of such basic compounds are those that form non-toxic acid addition salts, i.e., salts containing pharmacologically acceptable anions, including but not limited to, malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts.

Compounds included in the present compositions and methods that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts.

Compounds included in the present compositions and methods that include a basic or acidic moiety can also form pharmaceutically acceptable salts with various amino acids. The compounds of the disclosure can contain both acidic and basic groups; for example, one amino and one carboxylic acid group. In such a case, the compound can exist as an acid addition salt, a zwitterion, or a base salt.

The terms “pharmaceutically acceptable excipient,” “pharmaceutically acceptable diluent,” “pharmaceutically acceptable carrier,” and “pharmaceutically acceptable adjuvant” are used interchangeably and refer to an excipient, diluent, carrier, or adjuvant that is useful in preparing a pharmaceutical composition that are generally safe, non-toxic and neither biologically nor otherwise undesirable, and include an excipient, diluent, carrier, and adjuvant that are acceptable for veterinary use as well as human pharmaceutical use. The phrase “pharmaceutically acceptable excipient” includes both one and more than one such excipient, diluent, carrier, and/or adjuvant.

Generally, reference to or depiction of a certain element such as hydrogen or H is meant to include all isotopes of that element. For example, if an R group is defined to include hydrogen or H, it also includes deuterium and tritium. Compounds comprising radioisotopes such as tritium, 14C, 32P and 35S are thus within the scope of the present technology. Procedures for inserting such labels into the compounds of the present technology will be readily apparent to those skilled in the art based on the disclosure herein.

Unless the specific stereochemistry is expressly indicated, all chiral, diastereomeric, and racemic forms of a compound are intended. Thus, compounds described herein include enriched or resolved optical isomers at any or all asymmetric atoms as are apparent from the depictions. Racemic mixtures of R-enantiomer and S-enantiomer, and enantio-enriched stereomeric mixtures comprising of R- and S-enantiomers, as well as the individual optical isomers can be isolated or synthesized so as to be substantially free of their enantiomeric or diastereomeric partners, and these stereoisomers are all within the scope of the present technology.

The compounds described herein may exist as solvates, especially hydrates, and unless otherwise specified, all such solvates and hydrates are intended. Hydrates may form during manufacture of the compounds or compositions comprising the compounds, or hydrates may form over time due to the hygroscopic nature of the compounds. Compounds of the present technology may exist as organic solvates as well, including DMF, ether, and alcohol solvates, among others. The identification and preparation of any particular solvate is within the skill of the ordinary artisan of synthetic organic or medicinal chemistry.

As described herein, the text refers to various embodiments of the present compounds, compositions, and methods. The various embodiments described are meant to provide a variety of illustrative examples and should not be construed as descriptions of alternative species. Rather, it should be noted that the descriptions of various embodiments provided herein may be of overlapping scope. The embodiments discussed herein are merely illustrative and are not meant to limit the scope of the present technology.

### 5.2. Other Interpretational Conventions

Ranges: throughout this disclosure, various aspects of the invention are presented in a range format. Ranges include the recited endpoints. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6, should be considered to have specifically disclosed subranges such as from 1 to 3, from 1 to 4, from 1 to 5, from 2 to 4, from 2 to 6, from 3 to 6, etc. as well as individual number within that range, for example, 1, 2, 3, 4, 5, 5.3, and 6. This applies regardless of the breadth of the range.

In this disclosure, “comprises,” “comprising,” “containing,” “having,” “includes,” “including”, and linguistic variants thereof have the meaning ascribed to them in U.S. Patent law, permitting the presence of additional components beyond those explicitly recited.

Unless specifically stated or apparent from context, as used herein the term “or” is understood to be inclusive.

Unless specifically stated or apparent from context, as used herein, the terms “a”, “an”, and “the” are understood to be singular or plural. That is, the articles “a” and “an” are used herein to refer to one or to more than one (i.e., to at least one) of the grammatical object of the article. By way of example, “an element” means one element or more than one element.

Unless specifically stated or otherwise apparent from context, as used herein the term “about” is understood as within range of normal tolerance in the art, for example within 2 standard deviations of the mean, and is meant to encompass variations of ±20% or ±10%, more preferably ±5%, even more preferably ±1%, and still more preferably ±0.1% from the stated value. Where a percentage is provided with respect to an amount of a component or material in a composition, the percentage should be understood to be a percentage based on weight, unless otherwise stated or understood from the context.

It should be understood that two or more steps or actions can be conducted simultaneously.

### 5.3. Summary of Experimental Observations

As detailed in the experimental examples in this disclosure, we have shown that SOD1 mice, a model for amyotrophic lateral sclerosis (ALS), had a ˜5-fold higher percentage of Ly6C^{hi} inflammatory monocytes in their peripheral circulation than do wild-type mice (˜2% vs ˜11%). Treatment with 50 mg/kg TQS-168 administered by oral gavage every 3 days from Day 70 until Day 90 inhibited the increase in circulating Ly6C^{hi} monocytes in the SOD1 mice. SOD1 mice receiving TQS-168 by oral gavage survived longer than mice receiving the vehicle alone.

We next demonstrated that ALS patients analogously display an abnormally high percentage of CD14+CD16+ monocytes in the peripheral circulation as compared with healthy controls. We further demonstrated that a 4-hour ex vivo incubation of blood samples from ALS patients with TQS-168 caused a dose-dependent decrease in the percentage of intermediate (CD14+CD16+) monocytes in the samples, with the highest concentration of TQS-168, 30 μM, reducing CD14+CD16+ monocytes from 1.5% to less than 1%.

Collectively, these data indicate that circulating inflammatory myeloid cells, such as CD14+CD16+ monocytes in human patients, are a clinically accessible surrogate marker for the effects of 2-arylbenzimidazoles such as TQS-168 in treatment of neuroinflammatory and neurodegenerative disorders. The data indicate that circulating inflammatory myeloid cells, such as CD14+CD16+ monocytes, can be used to identify and select patients for treatment with TQS-168 and other 2-aryl-benzimidazoles and can be used to monitor and titrate treatment of such patients, including human ALS patients.

### 5.4. Methods of Selecting a Subject for Treatment with 2-Arylbenzimidazole Compounds

Accordingly, in a first aspect, methods are presented for selecting a subject for treatment with certain therapeutic 2-arylbenzimidazole compounds, as described in section 5.9 herein, or a salt, hydrate, deuterated analog, or fluorinated analog thereof. The method comprises the steps of:
a) measuring the baseline concentration of CD14+CD16+ myeloid cells in a biological sample containing circulating myeloid cells from the subject;
b) comparing the baseline concentration of circulating CD14+CD16+ myeloid cells to a predetermined threshold concentration; and
c) if the patient baseline concentration of CD14+CD16+ myeloid cells is greater than the predetermined threshold, thereby indicating a circulating myeloid cell inflammatory phenotype, selecting the patient for treatment.

In some embodiments, the CD14+CD16+ circulating myeloid cells are CD14+CD16+ circulating monocytes.

In typical embodiments, the biological sample is a whole blood sample drawn from the subject. In some embodiments, the biological sample is a cellular fraction derived from a whole blood sample.

In some embodiments of the method, the predetermined threshold is the concentration of circulating CD14+CD16+ myeloid cells in a biological sample containing circulating myeloid cells from a healthy individual. In typical embodiments, the biological sample providing the predetermined threshold value is a whole blood sample. In some embodiments, the biological sample providing the predetermined threshold value is a fraction derived from a whole blood sample of a healthy individual.

In some embodiments of the method, the healthy subject or individual is a person not diagnosed with a neurodegenerative disease. In some embodiments of the method, a healthy subject is a person not diagnosed with a neurodegenerative disease selected from amyotrophic lateral sclerosis (ALS), Alzheimer's Disease (Aβ), Lewy Body dementia, vascular or multi-infarct dementia, or frontotemporal dementia (FTD), Parkinson's disease, Huntington's disease, demyelinating disease, and multiple sclerosis (MS). In some embodiments of the method, a healthy subject is a person not diagnosed with ALS.

In some embodiments of the method, the predetermined threshold is the mean concentration of circulating CD14+CD16+ myeloid cells in biological samples containing circulating myeloid cells from a healthy population. In some embodiments of the method, the predetermined threshold is the median concentration of circulating CD14+CD16+ myeloid cells in biological samples containing circulating myeloid cells from a healthy population. In some embodiments of the method, the predetermined threshold is the maximum of the range of values of the percentage of circulating CD14+CD16+ myeloid cells in a healthy subject.

In some embodiments of the method, the predetermined threshold is a single value. In some embodiments of the method, the predetermined threshold is a range of values.

In some embodiments of the method, the predetermined threshold is the concentration of circulating CD14+CD16+ myeloid cells compared to total circulating myeloid cells. In various embodiments, the concentration is 0.85%, 0.86%, 0.88%, 0.89%, 0.90%, 0.91%, 0.92%, 0.93%, 0.94%, 0.95%, 0.96%, 0.97%, 0.98%, 0.99%, 1.0%, 1.01%, 1.02%, 1.03%, 1.04%, 1.05%, 1.06%, 1.07%, 1.08%, 1.09%, 1.10%, 1.11%, 1.12%, 1.13%, 1.14%, 1.15%, 1.16%, 1.17%, 1.18%, 1.19%, 1.20%, 1.21%, 1.22%, 1.23%, 1.24%, 1.25%, 1.26%, 1.27%, 1.28%, 1.29%, 1.30%, 1.31%, 1.32%, 1.33%, 1.34%, 1.35%, 1.36%, 1.37%, 1.38%, 1.39%, 1.40%, 1.41%, 1.42%, 1.43%, 1.44%, 1.45%, 1.46%, 1.47%, 1.48%, 1.49%, 1.50%, 1.51%, 1.52%, 1.53%, 1.54%, 1.55%, 1.56%, 1.57%, 1.58%, 1.59%, 1.60%, 1.61%, 1.62%, 1.63%, 1.64%, 1.65%, 1.66%, 1.67%, 1.68%, 1.69%, or 1.70%.

In some embodiments of the method, the predetermined threshold is greater than the median across a healthy population of men between the ages of at least 18 years old. In some further embodiments of the method, the men are 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 years old. In some further embodiments of the method, the predetermined threshold is greater than the median across a healthy population of men between the ages of at least 18˜40 years old.

In some embodiments of the method, the predetermined threshold is greater than the mean across a healthy population of men between the ages of at least 18 years old. In some further embodiments of the method, the men are 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 years old. In some further embodiments of the method, the predetermined threshold is greater than the mean across a healthy population of men between the ages of at least 18˜40 years old.

In some embodiments of the method, the predetermined threshold is greater than the median across a healthy population of women between the ages of at least 18 years old. In some further embodiments of the method, the women are 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 years old. In some further embodiments of the method, the predetermined threshold is greater than the median across a healthy population of women between the ages of at least 18-40 years old.

In some embodiments of the method, the predetermined threshold is greater than the mean across a healthy population of women between the ages of at least 18 years old. In some further embodiments of the method, the women are 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 years old. In some further embodiments of the method, the predetermined threshold is greater than the mean across a healthy population of women between the ages of at least 18-40 years old.

In some embodiments of the method, the healthy population is a mixture of men and women. In some embodiments of the method, the healthy population is a mixture of men and women at least 18 years old. In some further embodiments of the method, the men and women are 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 years old. In some embodiments of the method, the healthy population is a mixture of men and women between the ages of at least 18-40 years old.

In some embodiments of the method, the subject who is selected for treatment is administered a therapeutically effective amount of a compound of Formula I, as described in Section 5.9 herein, or a salt, hydrate, deuterated analog, or fluorinated analog thereof. In some embodiments of the method, the therapeutically effective amount is an amount sufficient to suppress the inflammatory phenotype of circulating myeloid cells. In some embodiments of the method, the therapeutically effective amount is effective to suppress synthesis, and optionally secretion, of one of more inflammatory cytokines upon exposure of the circulating inflammatory myeloid cells to LPS in vitro. In some embodiments, the one or more inflammatory cytokines are selected from IFN-γ, IL-6, TNF-α, MCP-1, and IL12.

In some embodiments of the method, the subject selected for treatment with the compound of Formula I or a salt, hydrate, deuterated analog, or fluorinated analog thereof has not previously been diagnosed with, but is at risk for developing, neuroinflammation.

In some embodiments of the method, the subject selected for treatment with the compound of Formula I or a salt, hydrate, deuterated analog, or fluorinated analog thereof has not previously been diagnosed with, but is at risk for developing, a neurodegenerative disease.

In some embodiments of the method, the neurodegenerative disease the subject has not previously been diagnosed with, but is at risk for developing, is amyotrophic lateral sclerosis (ALS), Alzheimer's Disease (Aβ), Lewy Body dementia, vascular or multi-infarct dementia, or frontotemporal dementia (FTD), Parkinson's disease, Huntington's disease, demyelinating disease, or multiple sclerosis (MS).

In some embodiments of the method, the subject has at least one ApoE4 allele. In particular embodiments of the method, the subject has two ApoE4 alleles. In some particular embodiments of the method, the method further comprises the prior step of detecting the presence of ApoE4 alleles in the subject.

In particular embodiments of the method, the subject has a family history of neurodegenerative disease. In some particular embodiments of the method, the neurodegenerative disease in the subject's family history is amyotrophic lateral sclerosis (ALS), Alzheimer's Disease (Aβ), Lewy Body dementia, vascular or multi-infarct dementia, or frontotemporal dementia (FTD), Parkinson's disease, Huntington's disease, demyelinating disease, or multiple sclerosis (MS).

In some embodiments of the method, the subject has a mild cognitive impairment. In certain embodiments, the diagnosis of MCI is made using the Short Test of Mental Status, the Montreal Cognitive Assessment (MoCA) or the Mini-Mental State Examination (MMSE).

In some embodiments of the method, the subject has TREM2 heterozygous or homozygous mutations. In some particular embodiments of the method, the subject has TREM2 heterozygous mutations. In some particular embodiments of the method, the subject has TREM2 homozygous mutations. In some embodiments of the method, TREM2 variants display partial loss of function of the TREM2 protein. In some embodiments of the method, TREM2 variants display abnormal function of the TREM2 protein. In some embodiments of the method, TREM2 variants alter the behavior of microglial cells, including their response to amyloid plaques. In some particular embodiments of the method, the method further comprises the prior step of detecting the presence of TREM2 heterozygous or homozygous mutations in the subject.

In some embodiments of the method, the subject has PSEN1 heterozygous or homozygous mutations. In some particular embodiments of the method, the subject has PSEN1 heterozygous mutations. In some particular embodiments of the method, the subject has PSEN1 homozygous mutations. In some embodiments of the method, PSEN1 variants display partial loss of function of the PSEN1 protein. In some embodiments of the method, PSEN1 variants display abnormal function of the PSEN1 protein. In some embodiments of the method, subjects with PSEN1 variants display increased amyloid plaque production. In some particular embodiments of the method, the method further comprises the prior step of detecting the presence of PSEN1 heterozygous or homozygous mutations in the subject.

In some embodiments of the method, the subject has positive amyloid-β (Aβ) or tau protein in cerebrospinal fluid (CSF). In some embodiments of the method, the subject has abnormal levels of total tau and phosphorylated tau in the CSF.

In some embodiments of the method, the subject has had a positive amyloid-β (Aβ) PET scan prior to treatment.

In some embodiments of the method, the subject has been determined prior to treatment to have at least one of (i) decreased levels of Aβ42 in plasma, (ii) increased levels of Aβ40 in plasma, (iii) decreased ratio of Aβ42/Aβ40 in plasma, (iv) increased levels of neurofilament light (NFL) in plasma, and (v) increased levels of neurogranin in plasma, as compared to a healthy control population that does not have neurodegenerative disease.

In some embodiments of the method, the subject has had a positive tau protein PET scan prior to treatment. In some particular embodiments of the method, the subject has been determined prior to treatment to have abnormal patterns of Tau based on Tau PET imaging.

In some embodiments of the method, the subject has GRN heterozygous or homozygous mutations. In some particular embodiments of the method, the subject has GRN heterozygous mutations. In some particular embodiments of the method, the subject has GRN homozygous mutations. In certain embodiments of the method, GRN variants display partial loss of function of the GRN protein. In certain embodiments of the method, GRN variants display abnormal function of the GRN protein. In some particular embodiments of the method, the method further comprises the prior step of detecting the presence of GRN heterozygous or homozygous mutations in the subject.

In some embodiments of the method, the subject has reduced progranulin levels. In some embodiments of the method, the subject has progranulin levels

In some embodiments of the method, the subject selected for treatment with the compound of Formula I or a salt, hydrate, deuterated analog, or fluorinated analog thereof, has not previously been diagnosed with cognitive impairment, but is at least 40 years old. In some embodiments of the method, the subject is at least 45 years old. In some embodiments of the method, the subject is at least 50 years old. In some embodiments of the method, the subject is at least 55 years old. In some embodiments of the method, the subject is at least 60 years old. In some embodiments of the method, the subject is at least 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 years old.

In some embodiments of the method, the subject is deselected for treatment if the baseline concentration of circulating CD14+CD16+ myeloid cells is less than the predetermined threshold, thereby indicating a circulating myeloid cell non-inflammatory phenotype.

### 5.5. Methods of Treating a Subject Who has not Previously been Diagnosed with, but is at Risk for Developing, Neuroinflammation and/or a Neurodegenerative Disease

In another aspect, methods are presented for treating a subject who has not previously been diagnosed with, but is at risk for developing, neuroinflammation and/or a neurodegenerative disease. The method comprises selecting a patient for treatment according to the methods described in Section 5.4 herein; and administering to the subject who is selected for treatment at least a first dose of a 2-arylbenzimidazole compound of Formula I as described in section 5.9 herein, or a salt, hydrate, deuterated analog, or fluorinated analog thereof.

In some particular embodiments, the method further comprises determining the post-dose concentration of circulating CD14+CD16+ myeloid cells in a biological sample obtained from the subject, comparing the subject's post-dose concentration to the subject's baseline concentration, and if the post-dose concentration is less than the baseline concentration, administering a plurality of subsequent doses at the same or at higher amounts.

In some particular embodiments, the method further comprises, if the post-dose concentration is not less than the baseline blood concentration, repeating the steps of determining the post-dose concentration of circulating CD14+CD16+ myeloid cells in a biological sample obtained from the subject, and comparing the subject's post-dose concentration to the subject's baseline concentration, and if the post-dose concentration is less than the baseline concentration, administering one or more successively increased doses of the compound of Formula I until either the subject's post-dose concentration of circulating CD14+CD16+ myeloid cells is lower than the subject's baseline concentration, or a predetermined maximum dose of the compound is reached.

In some further particular embodiments of the method, the method further comprises deselecting the subject for further treatment if no post-dose concentration of CD14+CD16+ myeloid cells is lower than the patient's baseline concentration.

### 5.6. Methods of Treating a Subject Who has not Previously been Diagnosed with Cognitive Impairment, but is at Least 40 Years Old

In a further aspect, methods are presented for treating a subject who has not previously been diagnosed with cognitive impairment, but is at least 40 years old. The method comprises: selecting a patient for treatment according to the methods described in section 5.4 herein; and administering to the subject who is selected for treatment at least a first dose of a 2-arylbenzamidazole compound of Formula (I) as described in section 5.9 herein, or a salt, hydrate, deuterated analog, or fluorinated analog thereof.

In some embodiments, the method further comprises determining the post-dose concentration of circulating CD14+CD16+ myeloid cells in a biological sample obtained from the subject, comparing the subject's post-dose concentration to the subject's baseline concentration, if the post-dose concentration is less than the baseline concentration, administering a plurality of subsequent doses at the same or at higher amounts.

In some further embodiments of the method, after determining the post-dose concentration of circulating CD14+CD16+ myeloid cells in a biological sample obtained from the subject, the method further comprises comparing the subject's post-dose concentration to the subject's baseline concentration, and if the post-dose concentration is less than the baseline concentration, administering a plurality of subsequent doses at the same or at higher amounts. In some embodiments, the methods further comprise, if the post-dose concentration is not less than the baseline blood concentration, repeating the above mentioned steps with successively increased doses of the compound of Formula I until either the subject's post-dose concentration of circulating CD14+CD16+ myeloid cells is lower than the subject's baseline concentration, or a predetermined maximum dose of the compound is reached.

In some further embodiments of the method, after determining the post-dose concentration of circulating CD14+CD16+ myeloid cells in a biological sample obtained from the subject, the method further comprises comparing the subject's post-dose concentration to the subject's baseline concentration, and if the post-dose concentration is less than the baseline concentration, administering a plurality of subsequent doses at the same or at higher amounts, and further, if the post-dose concentration is not less than the baseline blood concentration, repeating the above mentioned steps with successively increased doses of the compound of Formula I until either the subject's post-dose concentration of circulating CD14+CD16+ myeloid cells is lower than the subject's baseline concentration, or a predetermined maximum dose of the compound is reached, and further still, deselecting the subject for further treatment if no post-dose concentration of CD14+CD16+ myeloid cells is lower than the patient's baseline concentration.

### 5.7. Methods of Treating a Subject Who has an Inflammatory Phenotype of Circulating Myeloid Cells

In another aspect, methods are presented for treating a subject who has an inflammatory phenotype of circulating myeloid cells. The method comprises administering to the subject who has an inflammatory phenotype of circulating myeloid cells a therapeutically effective amount of a compound of Formula (I) as described in section 5.9 herein, or a salt, hydrate, deuterated analog, or fluorinated analog thereof.

In some embodiments of the method, the method further comprises determining the post-dose concentration of circulating CD14+CD16+ myeloid cells in a biological sample obtained from the subject, comparing the subject's post-dose concentration to the subject's baseline concentration, and if the post-dose concentration is less than the baseline concentration, administering a plurality of subsequent doses at the same or at higher amounts.

In another embodiment of the method, if the post-dose concentration is not less than the baseline blood concentration, the method further comprises repeating the steps of determining the post-dose concentration of circulating CD14+CD16+ myeloid cells in a biological sample obtained from the subject, comparing the subject's post-dose concentration to the subject's baseline concentration, and if the post-dose concentration is less than the baseline concentration, administering a plurality of subsequent doses at the same or at higher amounts with successively increased doses of the compound of Formula I until either the subject's post-dose concentration of circulating CD14+CD16+ myeloid cells is lower than the subject's baseline concentration, or a predetermined maximum dose of the compound is reached.

In another embodiment of the method, if the post-dose concentration is not less than the baseline blood concentration, the method comprises further administering a plurality of successively increased doses of the compound of Formula I until either the subject's post-dose concentration of circulating CD14+CD16+ myeloid cells is lower than the subject's baseline concentration, or a predetermined maximum dose of the compound is reached. If a maximum dose of the compound is reached without the subject's post-dose concentration of circulating CD14+CD16+ myeloid cells is lower than baseline, the method may further comprise deselecting the subject for further treatment if no post-dose concentration of CD14+CD16+ monocytes is lower than the patient's baseline concentration.

### 5.8. Methods of Assessing the Efficacy of a Compound in Treatment of Neurodegenerative Diseases

In another aspect, methods are provided for assessing the efficacy of a compound in treatment of neurodegenerative diseases, as set forth in the following numbered clauses.

1. A method of assessing efficacy of a compound in the treatment of neurodegenerative diseases in a human patient, by a 2-arylbenzimidazole moiety, the method comprising:
(a) determining a baseline expression level of one or more biomarkers in whole blood collected from healthy human subject(s);
(b) determining a baseline expression level of one or more of the same biomarkers utilized in step (a) in whole blood collected from patients suffering from one or more neurodegenerative F;
(c) comparing the baseline expression of the aforemeasured biomarkers in whole blood collected from healthy human subject(s) with the baseline expression level of the same biomarkers collected from the whole blood of patients suffering from one or more neurodegenerative diseases;
(d) administering a 2-arylbenzimidazole moiety to the patient suffering from one or more neurodegenerative diseases; and
(e) measuring the post-treatment change from baseline expression level of the biomarkers measured in step (b).

2. The method of embodiment 1, wherein the 2-arylbenzimidazole moiety is selected from TQS-168 and TQS-621.

3. The method of embodiment 1, wherein the biomarker is inflammatory phenotype.

4. The method of embodiment 1, wherein the inflammatory phenotype is CD14+CD16+.

5. The method of embodiment 1, wherein the neurodegenerative disorder is one or more of the following Alzheimer's Disease (Aβ), Parkinson's Disease (PD), Huntington's Disease (HD), Frontotemporal degeneration (FTD), Dementia with Lewy Bodies (DLB), Motor Neuron Diseases (MND), and Demyelinating Diseases.

6. A method of treating a neurodegenerative disease by administering a 2-arylbenzimidazole moiety to the patient, the method comprising:
(a) using the numbers of peripherally circulating CD14+ and CD16+ monocytes/macrophages; and/or
(b) the percentage reductions in circulating CD14 and CD16 monocytes; and/or
(c) the changes in CD14+/CD16+ ratios to determine by way of titration the appropriate therapeutic dosage for the patient.

### 5.9. 2-arylbenzimidazole compounds of Formula (I)

In the methods described herein, the 2-arylbenzimidazole compounds are compounds of Formula I,

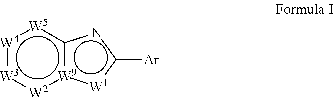

or a salt, hydrate, deuterated analog, or fluorinated analog thereof, wherein
Ar is:

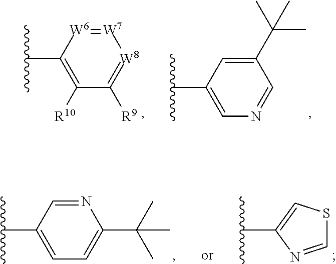

W¹ is chosen from N—R¹, O, and S, or when W⁹ is N, W¹ may additionally be C—R⁵⁰;
W² is C—R² or N;
W³ is C—R³ or N;
W⁴ is C—R⁴ or N;
W⁵ is C—R⁵ or N;
W⁶ is C—R⁶ or N;
W⁷ is C—R⁷ or N;
W⁸ is C—R⁸ or N;
W⁹ is C, or when W¹ is C—R⁵⁰, W⁹ may be N;
R¹ is selected from H, (C₁-C₃)alkyl, —CH₂OC(═O)R³⁰, —CH₂OP(═O)OR⁴⁰OR⁴¹, —C(═O)OR⁴², and —C(═O)R⁴³;
R², R³, R⁴, and R⁵ are selected independently from hydrogen, deuterium, halogen, perfluoro(C₁-C₄)alkyl, (C₁-C₄)alkyl, (C₁-C₄)alkoxy, perfluoro(C₁-C₄)alkoxy, (C₁-C₄)acyl, (C₁-C₄)alkoxy(C₁-C₄)alkyl, hydroxy(C₁-C₄)alkyl, hydroxy, carboxy, (C₁-C₄)alkoxycarbonylamino, carboxamido, (C₁-C₄)alkylaminocarbonyl, cyano, acetoxy, nitro, amino, (C₁-C₄)alkylamino, di(C₁-C₄)alkylamino, mercapto, (C₁-C₄)alkylthio, aminosulfonyl, (C₁-C₄)alkylsulfonyl, and (C₁-C₄)acylamino;
R⁶ and R¹⁰ are selected independently from hydrogen, deuterium, halo, (C₁-C₃)alkyl, perfluoro(C₁-C₃)alkyl, hydroxy, (C₁-C₃)alkoxy, perfluoro(C₁-C₃)alkoxy, and amino;
R⁷ and R⁹ are selected independently from hydrogen, deuterium, hydroxy, cyano, amino, halogen, halo(C₁-C₄)alkyl, (C₁-C₄)alkoxy, halo(C₁-C₄)alkoxy,

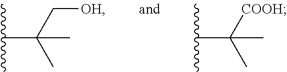

R⁸ is selected from hydrogen, deuterium, halogen, halo(C₁-C₄)alkyl, (C₁-C₄)alkyl, (C₁-C₄)alkoxy, halo(C₁-C₄)alkoxy, cyano, phenyl, phenoxy, benzyloxy, amino,

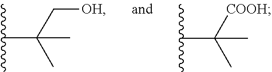

R³⁰ is selected from (C₁-C₁₀)hydrocarbyl, (C₁-C₁₀)hydrocarbyl substituted with amino, (C₁-C₁₀)hydrocarbyl substituted with (C₁-C₄)hydrocarbyl, (C₁-C₁₀)hydrocarbyl substituted with carboxyl, carboxy, (C₁-C₆)alkoxycarbonyl, (C₁-C₆)alkoxycarbonylamino, methylthio, heterocyclyl, (C₁-C₁₀)oxaalkyl, CHR⁴⁴NHR⁴⁵ and guanidine;
R⁴⁰ and R⁴¹ are selected independently from hydrogen (C₁-C₆)hydrocarbyl;
R⁴² is (C₁-C₅)alkyl;
R⁴³ is (C₁-C₃)alkyl,
R⁴⁴ is selected from any naturally occurring amino acid sidechain;
R⁴⁵ is selected from H, methyl, and (C₁-C₄)alkoxycarbonyl; and
R⁵⁰ is H or (C₁-C₃)alkyl.

In some embodiments, the compound of Formula I is selected from:

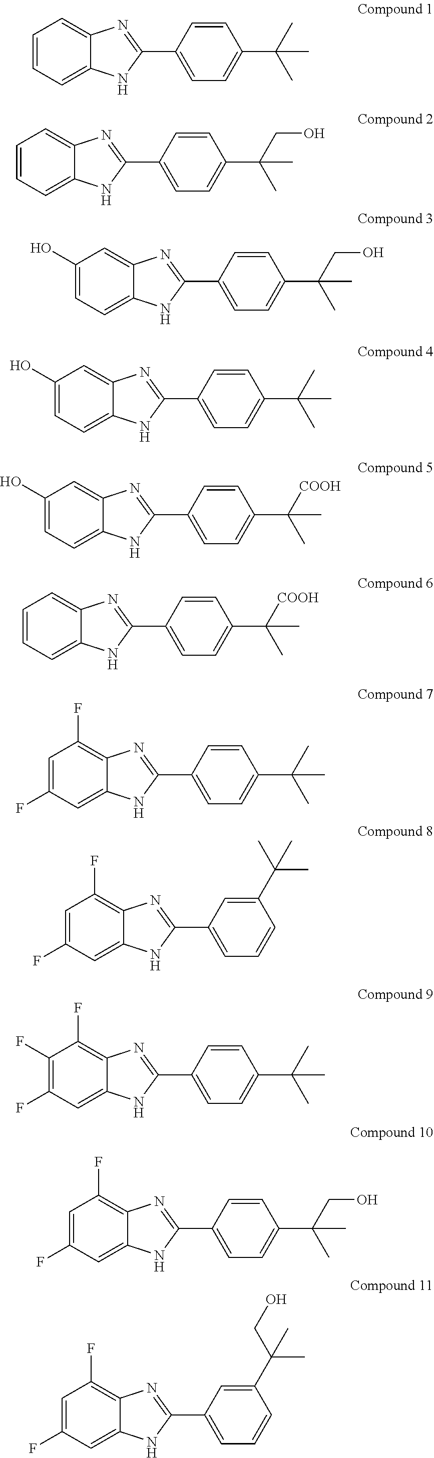

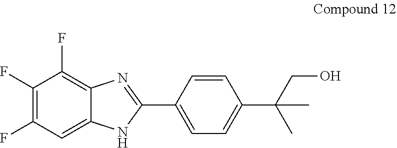 [SMILES: CC(C)(CO)c1ccc(-c2nc3c(F)c(F)c(F)cc3[nH]2)cc1]

or a salt, hydrate, deuterated analog, or fluorinated analog thereof.

In certain embodiments, the compound of Formula (I) is compound 1

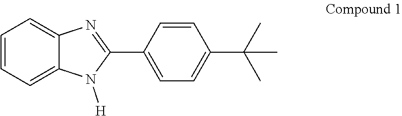 [SMILES: CC(C)(C)c1ccc(-c2nc3ccccc3[nH]2)cc1]

(also known as ZLN-005 or TQS-168), or a salt, hydrate, deuterated analog, or fluorinated analog thereof.

In certain embodiments, the compound of Formula (I) is compound 4

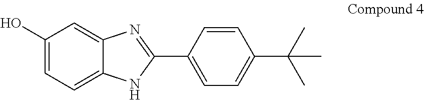 [SMILES: CC(C)(C)c1ccc(-c2nc3cc(O)ccc3[nH]2)cc1]

(also known as TQS-621), or a salt, hydrate, deuterated analog, or fluorinated analog thereof.

### 5.10. Measuring Circulating Myeloid Cells

In typical embodiments of the methods described herein, the circulating CD14+CD16+ myeloid cells are monocytes.

In certain embodiments of the methods described herein, the biological sample is whole blood. In certain embodiments, the biological sample is a compartment or fraction of whole blood.

In certain embodiments, the concentration of CD14+CD16+ circulating myeloid cells is determined by measuring at least one of CD14 and CD16 polypeptide, CD14 and CD16 mRNA, and CD14 and CD16 cDNA in the biological sample.

In certain embodiments, wherein the concentration of CD14+CD16+ monocytes is determined by measuring CD14 and CD16 polypeptide.

In certain embodiments, the concentration of CD14+CD16+ monocytes in the sample is measured by using one or more reagents that specifically bind CD14 or CD16 polypeptide.

In the methods described herein, in certain embodiments the reagent is selected from the group consisting of an antibody, an antibody derivative, and an antigen-binding antibody fragment. In certain embodiments, the antibody, antibody derivative, antigen-binding antibody fragment may be an IgA, IgD, IgE, IgG or IgM antibody, including any subclass of these isotypes. In certain embodiments, the antibody is an IgG antibody.

In the methods described herein, in certain embodiments, the concentration of CD14+CD16+ monocytes is determined by flow cytometry.

In the methods described herein, in certain embodiments the levels of CD14 and CD16 polypeptide are determined by detecting the amount of a transcribed polynucleotide or portion thereof. In the methods described herein, in certain embodiments, the transcribed polynucleotide is an mRNA. In the methods described herein, in certain embodiments, the transcribed polynucleotide is a cDNA. In the methods described herein, in certain embodiments, the detecting further comprises amplifying the transcribed polynucleotide or portion thereof.

### 5.11. Dose Regimens and Dosage Forms

In certain embodiments of the methods described herein in which a compound of Formula I, or a salt, hydrate, deuterated analog, or fluorinated analog thereof, the compound is administered orally.

In some embodiments, the compound is administered as a tablet.

In certain embodiments, the administered dose is about 200 mg to about 800 mg.

In the methods described herein, in certain embodiments, the administered dose is about 300 mg to about 700 mg.

In the methods described herein, in certain embodiments, the administered dose ranges is about 400 mg to about 600 mg.

In the methods described herein, in certain embodiments, the dose is 400 mg, 450 mg, or 500 mg.

In the methods described herein, in certain embodiments, the administered dose is about 100 mg, 120 mg, 140 mg, 160 mg, 180 mg, 200 mg, 220 mg, 240 mg, 260 mg, 280 mg, 300 mg, 320 mg, 340 mg, 360 mg, 380 mg, 400 mg, 420 mg, 440 mg, 460 mg, 480 mg, 500 mg, 520 mg, 540 mg, 560 mg, 580 mg, 600 mg, 620 mg, 640 mg, 660 mg, 680 mg, or 700 mg.

In the methods described herein, in certain embodiments, the administered dose is 100 mg, 120 mg, 140 mg, 160 mg, 180 mg, 200 mg, 220 mg, 240 mg, 260 mg, 280 mg, 300 mg, 320 mg, 340 mg, 360 mg, 380 mg, 400 mg, 420 mg, 440 mg, 460 mg, 480 mg, 500 mg, 520 mg, 540 mg, 560 mg, 580 mg, 600 mg, 620 mg, 640 mg, 660 mg, 680 mg, or 700 mg.

In the methods described herein, in certain embodiments, the dose is administered daily.

In the methods described herein, in certain embodiments, the dose is administered once daily as a single undivided dose.

In certain embodiments, the dose is administered as a plurality of divided doses. In certain embodiments, the dose is administered every other day.

### 6. EXAMPLES

It will be appreciated that various changes can be made without departing from the spirit and scope of the invention(s). Many variations will become apparent to those skilled in the art upon review of this specification.

### 6.1. Example 1—TQS-168 Suppresses the Inflammatory Effects of LPS on Circulating Myeloid Cells from Wild Type Mice (Ex Vivo)

Lipopolysaccharide (LPS) was used to induce circulating myeloid cells from wild type mice to adopt an inflammatory phenotype in vitro. Cells were then incubated in vitro with TQS-168 (2-(4-tert-butylphenyl)˜1H-benzimidazole) to assess whether TQS-168 could suppress the development of the LPS-promoted inflammatory phenotype in ex vivo cell cultures.

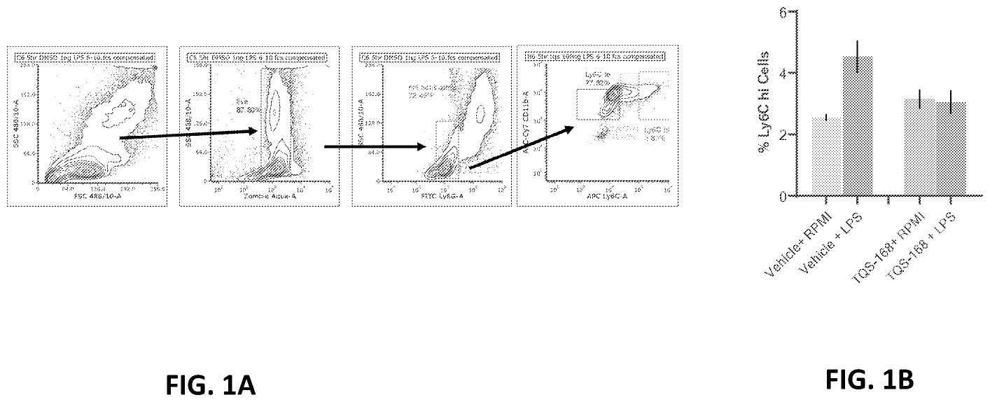

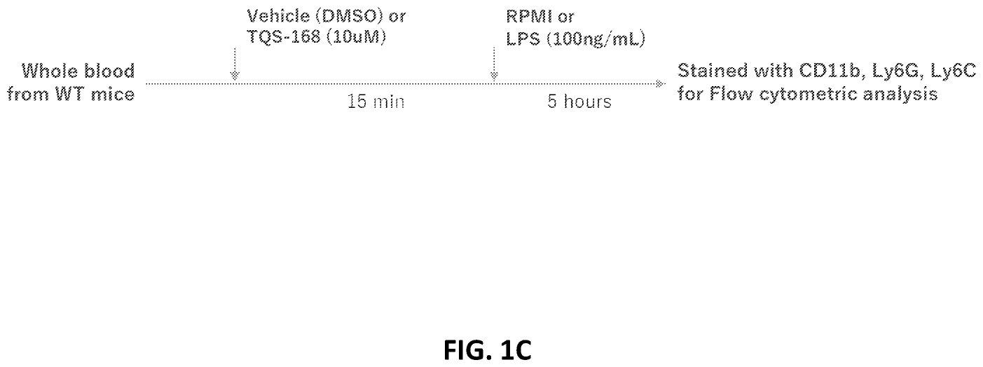

As schematized in FIG. 1C, blood samples from wild-type mice were collected in EDTA tubes and stimulated with LPS. Whole blood was incubated at 37° C. with increasing concentrations of TQS-168 for 5 hours. Whole blood was lysed with lysis buffer from BD Bioscience for 10 minutes at 37C. Lysed blood was centrifuged at 500 g for 10 minutes. The supernatant was decanted, and cell pellet washed with Hank's balanced salt solution (HBSS).

The cells were then centrifuged at 300 g for 10 minutes. They were subsequently resuspended in 2 mM EDTA FACS buffer. Fixed cells (100 μL) were washed with PBS. The cells (100 μL) were resuspended in PBS containing Zombie Aqua solution (Biolegend) at a 1:500 dilution. The cells were incubated in the dark at room temperature for 5 minutes. The cells were then washed with FACS buffer. The cells were stained with Cd11b, Ly6G, and Ly6C antibodies (BD Bioscience). The cells were then incubated with antibodies for 30 minutes. They were then washed with FACS buffer and then resuspended also in FACS buffer. Finally, the cells were acquired on flow cytometer.

As shown in FIG. 1B, LPS stimulation led to a 2-fold increase in the percentage of Ly6C^{hi} inflammatory monocytes, from about 2.5% to about 5%. Pre-incubation with TQS-168 inhibits the LPS-induced increase in Ly6C^{hi} monocytes, indicating that TQS-168 inhibits the acute inflammatory response to LPS.

### 6.2. Example 2—TQS-168 is Orally Bioavailable in Mice

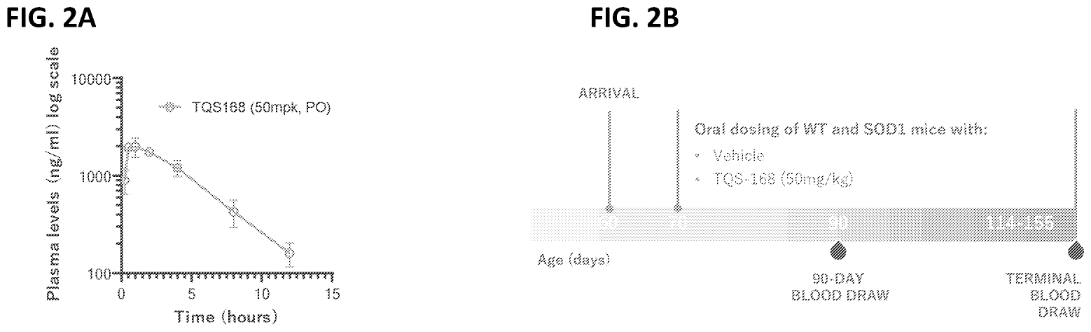

Plasma levels of TQS-168 in wild-type mice were analyzed subsequently to a single 50 mg/kg dose of TQS-168. The results of the pharmacokinetic study indicate that TQS-168 is orally bioavailable in mice. Results are shown in FIG. 2A.

### 6.3. Example 3—TQS-168 Suppresses Inflammatory Markers in SOD1 Mice

### 6.3.1. TQS-168 Reduces the Fraction of Inflammatory Monocytes in SOD1 Mice

The SOD1-G93A transgenic mouse has proven to be a useful tool in the study of ALS. A large majority of ALS cases are due to familial genetic mutations in the Superoxide dismutase 1 gene. SOD1-G93A mice express human SOD1 with the G93A mutation under control of the human SOD1 promoter. The mice show a phenotype similar to Amyotrophic Lateral Sclerosis in humans.

60-day old transgenic mice overexpressing the human SOD1-G93A ALS-causing mutation (stock #002726) and wild-type mice (WT; C₅₇BL/6J) were obtained from the Jackson Labs. Oral dosing by gavage started at 70 days of age. SOD1 mice received either TQS-168 50 mg/kg every 3 days or vehicle (same volume and frequency). The staff that dosed and assessed the mice were blinded to treatment allocation. Blood samples were collected in EDTA tubes at 2 time points: 90 days of age and time of euthanasia. Blood samples were processed for flow cytometry and plasma cytokine analysis. Flow cytometry methods employed are the same as those employed in Example 1 and Example 2.

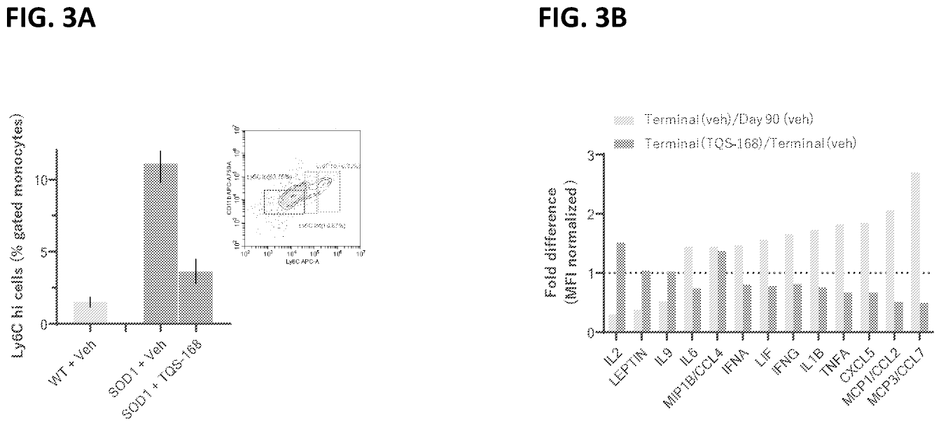

The results in FIG. 3A indicate SOD1 mice had a ˜5-fold higher percentage of Ly6C^{hi} inflammatory monocytes than wild-type mice (˜2% vs ˜11%). This increase is consistent with the increase in inflammatory cytokines that occur with disease progression. Treatment with 50 mg/kg TQS-168 administered by oral gavage every 3 days from Day 70 until Day 90 inhibited the increase in Ly6C^{hi} monocytes in SOD1 mice, demonstrating that TQS-168 can inhibit the chronic inflammatory response in monocytes in SOD1 mice.

### 6.3.2. TQS-168 Regulates Plasma Levels of Anti-Inflammatory and Pro-Inflammatory Cytokines in SOD1 Mice

Blood samples were collected from SOD1 mice at Day 90 and time of euthanasia. Plasma samples were assayed using the 48-plex Procarta panel (Thermo Fisher).

FIG. 3B shows that plasma levels of multiple cytokines (IL-6, IL-10, TNFα, interferon gamma, CXCL5, etc.) increased from Day 90 to the day of euthanasia as the disease progressed in the SOD1 mice. Treatment with TQS-168 50 mg/kg by oral gavage every 3 days from Day 70 until the day of euthanasia inhibited this increase in plasma cytokines, demonstrating that TQS-168 has broad anti-inflammatory effects in the SOD1 mouse model of ALS.

### 6.3.3. TQS-168 Improves Survival in SOD1 Mice

60-day old transgenic mice overexpressing the human SOD1-G93A ALS-causing mutation (stock #002726) were obtained from the Jackson Labs. Dosing started at 70 days of age. SOD1 mice received either TQS-168 50 mg/kg every 3 days or vehicle (same volume and frequency) by oral gavage. The righting reflex (ability to turn over when place on their backs) was assessed every 3 days when mice were <110 days old and every day after age 110 days. Mice were euthanized if they could not right themselves in 20 seconds after being placed onto their back. Time from birth to euthanasia was calculated for each mouse and compared in the two groups.

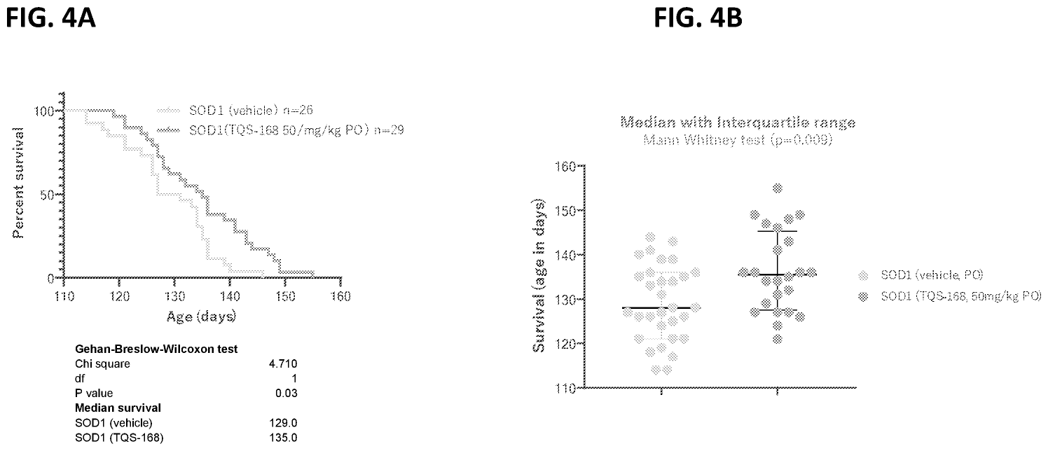

The mice receiving TQS-168 survived longer than mice receiving the vehicle. The difference in median survival was 6 days (129 in vehicle and 135 in TQS-168). The results displayed in FIGS. 4A-B demonstrate the ability of TQS-168 to increase survival in a model in which approved treatments for ALS, such as riluzole and edaravone, have failed to display survival benefits.

### 6.4. TQS-168 Suppresses Inflammatory Phenotype of Circulating Myeloid Cells from ALS Patients

### 6.4.1. Circulating CD14+CD16+ Cells are Increased in ALS Patients

Whole blood samples from patients with ALS were shipped at room temperature. The percentage of CD14+CD16+ monocytes were determined by fluorescence activated cell sorting (FACS).

Briefly, whole blood was lysed with lysis buffer (BD Bioscience) for 10 minutes at 37° C. Lysed blood was centrifuged at 500 g for 10 min. Supernatant was decanted and the cell pellet washed with HBSS (Hank's balanced salt solution). Cells were centrifuged at 300 g for 10 min. Cells were resuspended in FACS buffer (PBS+2% FBS=2 mM EDTA). Fixed cells (100 μL) were washed with PBS. Cells (100 μL) were resuspended in PBS containing Zombie Aqua (Biolegend; 1:500 dilution). Cells were incubated in the dark at room temperature for 5 minutes. Cells were washed with FACS buffer. Cells were stained with 5 μL CD16 and 5 μL CD14 antibodies (BD Bioscience). Cells were incubated with antibodies for 30 minutes. Cells were washed with FACS buffer. Cells were resuspended in FACS buffer. Cells were acquired on a flow cytometer.

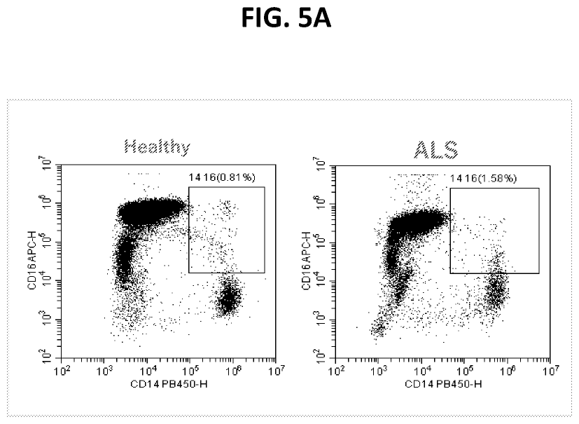

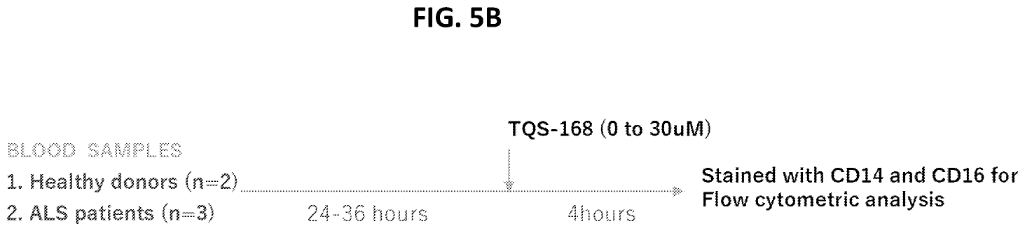

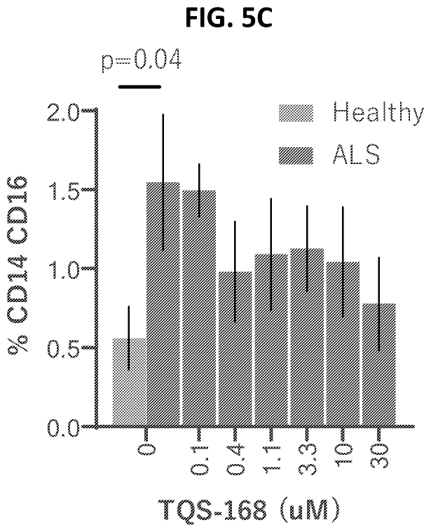

Human monocytes can be divided into 3 main subsets: classical (CD14^{hi} CD16^{lo}), intermediate (CD14^{hi} CD16^{hi}) and non-classical (CD141° C. D16^{hi}). The intermediate monocytes (which are also routinely called CD14+CD16+) are considered pro-inflammatory and increase in certain diseases states. As shown in FIG. 5A, in our experiments healthy donors (n=2) had ˜0.5% intermediate monocytes (of the total monocyte population) while patients with ALS (n=3) had ˜1.5%.

### 6.4.2. TQS-168 Reduces Inflammatory CD14+CD16+ Monocyte Expression

FIG. 5B outlines the experimental protocol for assessing the effects of ex vivo incubation with TQS-168 on the inflammatory phenotype of circulating myeloid cells in peripheral blood samples of ALS patients. Samples were obtained from iSpecimen (Lexington, MA) and BioIVT (Westbury, NY). Blood samples were processed within 36 hours of collection and were never frozen. FACS analysis was conducted essentially as described above in Section 6.4.1.

FIG. 5C is a bar graph showing the results of a four-hour ex vivo incubation of the peripheral blood samples of ALS patients with increasing concentrations of TQS-168. A four-hour incubation with TQS-168 caused a dose-dependent decrease in the percentage of intermediate (CD14+CD16+) monocytes in the whole blood of ALS patients, with the highest concentration of TQS-168, 30 μM, reducing CD14+CD16+ monocytes from 1.5% to less than 1%.

This effect is consistent with the in vivo effects observed in Example 3, in which oral administration of TQS-168 to SOD1 mice caused a decrease in inflammatory monocytes in the peripheral circulation as compared to vehicle-treated mice (FIG. 3A).

### 7. Equivalents and Incorporation by Reference

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

1. A method of selecting a subject for treatment with a compound of Formula I 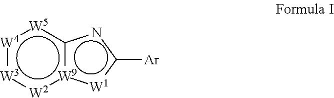
or a salt, hydrate, deuterated analog, or fluorinated analog thereof, wherein
Ar is: 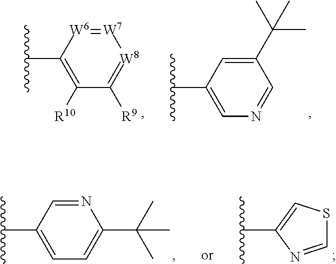
W¹ is chosen from N—R¹, O, and S, or when W⁹ is N, W¹ may additionally be C—R⁵⁰;
W² is C—R² or N;
W³ is C—R³ or N;
W⁴ is C—R⁴ or N;
W⁵ is C—R⁵ or N;
W⁶ is C—R⁶ or N;
W⁷ is C—R⁷ or N;
W⁸ is C—R⁸ or N;
W⁹ is C, or when W¹ is C—R⁵⁰, W⁹ may be N;
R¹ is selected from H, (C₁-C₃)alkyl, —CH₂OC(═O)R³⁰, —CH₂OP(═O)OR⁴⁰OR⁴¹, —C(═O)OR⁴², and —C(═O)R⁴³;
R², R³, R⁴, and R⁵ are selected independently from hydrogen, deuterium, halogen, perfluoro(C₁-C₄)alkyl, (C₁-C₄)alkyl, (C₁-C₄)alkoxy, perfluoro(C₁-C₄)alkoxy, (C₁-C₄)acyl, (C₁-C₄)alkoxy(C₁-C₄)alkyl, hydroxy(C₁-C₄)alkyl, hydroxy, carboxy, (C₁-C₄)alkoxycarbonylamino, carboxamido, (C₁-C₄)alkylaminocarbonyl, cyano, acetoxy, nitro, amino, (C₁-C₄)alkylamino, di(C₁-C₄)alkylamino, mercapto, (C₁-C₄)alkylthio, aminosulfonyl, (C₁-C₄)alkylsulfonyl, and (C₁-C₄)acylamino;
R⁶ and R¹⁰ are selected independently from hydrogen, deuterium, halo, (C₁-C₃)alkyl, perfluoro(C₁-C₃)alkyl, hydroxy, (C₁-C₃)alkoxy, perfluoro(C₁-C₃)alkoxy, and amino;
R⁷ and R⁹ are selected independently from hydrogen, deuterium, hydroxy, cyano, amino, halogen, halo(C₁-C₄)alkyl, (C₁-C₄)alkoxy, halo(C₁-C₄)alkoxy, 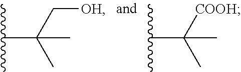
R⁸ is selected from hydrogen, deuterium, halogen, halo(C₁-C₄)alkyl, (C₁-C₄)alkyl, (C₁-C₄)alkoxy, halo(C₁-C₄)alkoxy, cyano, phenyl, phenoxy, benzyloxy, amino, 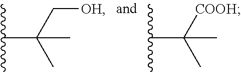
R³⁰ is selected from (C₁-C₁₀)hydrocarbyl, (C₁-C₁₀)hydrocarbyl substituted with amino, (C₁-C₁₀)hydrocarbyl substituted with (C₁-C₄)hydrocarbyl, (C₁-C₁₀)hydrocarbyl substituted with carboxyl, carboxy, (C₁-C₆)alkoxycarbonyl, (C₁-C₆)alkoxycarbonylamino, methylthio, heterocyclyl, (C₁-C₁₀)oxaalkyl, CHR⁴⁴NHR⁴⁵ and guanidine;
R⁴⁰ and R⁴¹ are selected independently from hydrogen (C₁-C₆)hydrocarbyl;
R⁴² is (C₁-C₅)alkyl;
R⁴³ is (C₁-C₃)alkyl,
R⁴⁴ is selected from any naturally occurring amino acid sidechain;
R⁴⁵ is selected from H, methyl, and (C₁-C₄)alkoxycarbonyl; and
R⁵⁰ is H or (C₁-C₃)alkyl;
the method comprising the steps of:
d) measuring the baseline concentration of CD14+CD16+ myeloid cells in a biological sample containing circulating myeloid cells from the subject;
e) comparing the baseline concentration of circulating CD14+CD16+ myeloid cells to a predetermined threshold concentration; and
f) if the patient baseline concentration of CD14+CD16+ myeloid cells is greater than the predetermined threshold, thereby indicating a circulating myeloid cell inflammatory phenotype, selecting the patient for treatment.

2. The method of claim 1, further comprising deselecting the subject for treatment if the baseline concentration of circulating CD14+CD16+ myeloid cells is less than the predetermined threshold, thereby indicating a circulating myeloid cell noninflammatory phenotype.

3. The method of claim 1, wherein the predetermined threshold is the concentration of circulating CD14+CD16+ myeloid cells in a biological sample containing circulating myeloid cells from a healthy individual.

4. The method of claim 1, further comprising administering to the subject who is selected for treatment a therapeutically effective amount of a compound of Formula I or a salt, hydrate, deuterated analog, or fluorinated analog thereof.

5. The method of claim 4, wherein the therapeutically effective amount is an amount sufficient to suppress the inflammatory phenotype of circulating myeloid cells.

6. The method of claim 1, wherein the subject selected for treatment with the compound of Formula I or a salt, hydrate, deuterated analog, or fluorinated analog thereof has not previously been diagnosed with, but is at risk for developing, neuroinflammation.

7. The method of claim 1, wherein the subject selected for treatment with the compound of Formula I or a salt, hydrate, deuterated analog, or fluorinated analog thereof has not previously been diagnosed with, but is at risk for developing, a neurodegenerative disease.

8. The method of claim 7, wherein the subject has at least one ApoE4 allele.

9. The method of claim 7, wherein the subject has family history of neurodegenerative disease, has mild cognitive impairment, TREM2 heterozygous or homozygous mutations, positive amyloid-β (Aβ) or tau protein in cerebrospinal fluid (CSF), had a positive amyloid-β (Aβ) PET scan, has GRN heterozygous or homozygous mutations, or reduced progranulin levels.

10-15. (canceled)

16. The method of claim 1, wherein the subject selected for treatment with the compound of Formula I or a salt, hydrate, deuterated analog, or fluorinated analog thereof, has not previously been diagnosed with cognitive impairment, but is at least 40 years old.

17. The method of claim 16, wherein the subject is at least 45 years old.

18-23. (canceled)

24. A method of treating a subject who has not previously been diagnosed with, but is at risk for developing, neuroinflammation and/or a neurodegenerative disease, the method comprising:
a) selecting a patient for treatment according to the method of claim 1; and
b) administering to the subject who is selected for treatment at least a first dose of a compound of Formula I 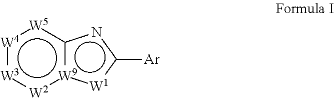
or a salt, hydrate, deuterated analog, or fluorinated analog thereof, wherein
Ar is: 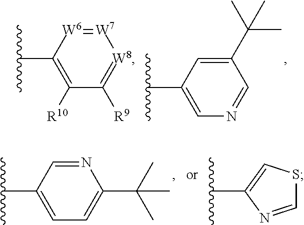
W¹ is chosen from N—R¹, O, and S, or when W⁹ is N, W¹ may additionally be C—R⁵⁰;
W² is C—R² or N;
W³ is C—R³ or N;
W⁴ is C—R⁴ or N;
W⁵ is C—R⁵ or N;
W⁶ is C—R⁶ or N;
W⁷ is C—R⁷ or N;
W⁸ is C—R⁸ or N;
W⁹ is C, or when W¹ is C—R⁵⁰, W⁹ may be N;
R¹ is selected from H, (C₁-C₃)alkyl, —CH₂OC(═O)R³⁰, —CH₂OP(═O)OR⁴⁰OR⁴¹, —C(═O)OR⁴², and —C(═O)R⁴³;
R², R³, R⁴, and R⁵ are selected independently from hydrogen, deuterium, halogen, perfluoro(C₁-C₄)alkyl, (C₁-C₄)alkyl, (C₁-C₄)alkoxy, perfluoro(C₁-C₄)alkoxy, (C₁-C₄)acyl, (C₁-C₄)alkoxy(C₁-C₄)alkyl, hydroxy(C₁-C₄)alkyl, hydroxy, carboxy, (C₁-C₄)alkoxycarbonylamino, carboxamido, (C₁-C₄)alkylaminocarbonyl, cyano, acetoxy, nitro, amino, (C₁-C₄)alkylamino, di(C₁-C₄)alkylamino, mercapto, (C₁-C₄)alkylthio, aminosulfonyl, (C₁-C₄)alkylsulfonyl, and (C₁-C₄)acylamino;
R⁶ and R¹⁰ are selected independently from hydrogen, deuterium, halo, (C₁-C₃)alkyl, perfluoro(C₁-C₃)alkyl, hydroxy, (C₁-C₃)alkoxy, perfluoro(C₁-C₃)alkoxy, and amino;
R⁷ and R⁹ are selected independently from hydrogen, deuterium, hydroxy, cyano, amino, halogen, halo(C₁-C₄)alkyl, (C₁-C₄)alkoxy, halo(C₁-C₄)alkoxy, 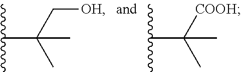
R⁸ is selected from hydrogen, deuterium, halogen, halo(C₁-C₄)alkyl, (C₁-C₄)alkyl, (C₁-C₄)alkoxy, halo(C₁-C₄)alkoxy, cyano, phenyl, phenoxy, benzyloxy, amino, 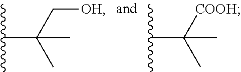
R³⁰ is selected from (C₁-C₁₀)hydrocarbyl, (C₁-C₁₀)hydrocarbyl substituted with amino, (C₁-C₁₀)hydrocarbyl substituted with (C₁-C₄)hydrocarbyl, (C₁-C₁₀)hydrocarbyl substituted with carboxyl, carboxy, (C₁-C₆)alkoxycarbonyl, (C₁-C₆)alkoxycarbonylamino, methylthio, heterocyclyl, (C₁-C₁₀)oxaalkyl, CHR⁴⁴NHR⁴⁵ and guanidine;
R⁴⁰ and R⁴¹ are selected independently from hydrogen (C₁-C₆)hydrocarbyl;
R⁴² is (C₁-C₅)alkyl;
R⁴³ is (C₁-C₃)alkyl,
R⁴⁴ is selected from any naturally occurring amino acid sidechain;
R⁴⁵ is selected from H, methyl, and (C₁-C₄)alkoxycarbonyl; and
R⁵⁰ is H or (C₁-C₃)alkyl.

25-27. (canceled)

28. A method of treating a subject who has not previously been diagnosed with cognitive impairment, but is at least 40 years old, the method comprising:
a) selecting a patient for treatment according to the method of claim 1; and
b) administering to the subject who is selected for treatment at least a first dose of a compound of Formula I 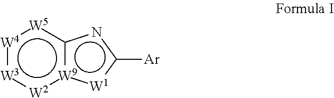
or a salt, solvate, hydrate, deuterated analog, or fluorinated analog thereof, wherein
Ar is: 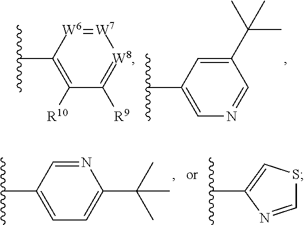
W¹ is chosen from N—R¹, O, and S, or when W⁹ is N, W¹ may additionally be C—R⁵⁰;
W² is C—R² or N;
W³ is C—R³ or N;
W⁴ is C—R⁴ or N;
W⁵ is C—R⁵ or N;
W⁶ is C—R⁶ or N;
W⁷ is C—R⁷ or N;
W⁸ is C—R⁸ or N;
W⁹ is C, or when W¹ is C—R⁵⁰, W⁹ may be N;
R¹ is selected from H, (C₁-C₃)alkyl, —CH₂OC(═O)R³⁰, —CH₂OP(═O)OR⁴⁰OR⁴¹, —C(═O)OR⁴², and —C(═O)R⁴³;
R², R³, R⁴, and R⁵ are selected independently from hydrogen, deuterium, halogen, perfluoro(C₁-C₄)alkyl, (C₁-C₄)alkyl, (C₁-C₄)alkoxy, perfluoro(C₁-C₄)alkoxy, (C₁-C₄)acyl, (C₁-C₄)alkoxy(C₁-C₄)alkyl, hydroxy(C₁-C₄)alkyl, hydroxy, carboxy, (C₁-C₄)alkoxycarbonylamino, carboxamido, (C₁-C₄)alkylaminocarbonyl, cyano, acetoxy, nitro, amino, (C₁-C₄)alkylamino, di(C₁-C₄)alkylamino, mercapto, (C₁-C₄)alkylthio, aminosulfonyl, (C₁-C₄)alkylsulfonyl, and (C₁-C₄)acylamino;
R⁶ and R¹⁰ are selected independently from hydrogen, deuterium, halo, (C₁-C₃)alkyl, perfluoro(C₁-C₃)alkyl, hydroxy, (C₁-C₃)alkoxy, perfluoro(C₁-C₃)alkoxy, and amino;
R⁷ and R⁹ are selected independently from hydrogen, deuterium, hydroxy, cyano, amino, halogen, halo(C₁-C₄)alkyl, (C₁-C₄)alkoxy, halo(C₁-C₄)alkoxy, 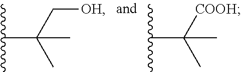
R⁸ is selected from hydrogen, deuterium, halogen, halo(C₁-C₄)alkyl, (C₁-C₄)alkyl, (C₁-C₄)alkoxy, halo(C₁-C₄)alkoxy, cyano, phenyl, phenoxy, benzyloxy, amino, 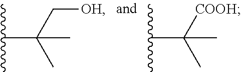
R³⁰ is selected from (C₁-C₁₀)hydrocarbyl, (C₁-C₁₀)hydrocarbyl substituted with amino, (C₁-C₁₀)hydrocarbyl substituted with (C₁-C₄)hydrocarbyl, (C₁-C₁₀)hydrocarbyl substituted with carboxyl, carboxy, (C₁-C₆)alkoxycarbonyl, (C₁-C₆)alkoxycarbonylamino, methylthio, heterocyclyl, (C₁-C₁₀)oxaalkyl, CHR⁴⁴NHR⁴⁵ and guanidine;
R⁴⁰ and R⁴¹ are selected independently from hydrogen (C₁-C₆)hydrocarbyl;
R⁴² is (C₁-C₅)alkyl;
R⁴³ is (C₁-C₃)alkyl,
R⁴⁴ is selected from any naturally occurring amino acid sidechain;
R⁴⁵ is selected from H, methyl, and (C₁-C₄)alkoxycarbonyl; and
R⁵⁰ is H or (C₁-C₃)alkyl.

29-31. (canceled)

32. A method of treating a subject who has an inflammatory phenotype of circulating myeloid cells, comprising:
b) administering to the subject a therapeutically effective amount of a compound of Formula I 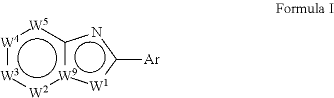
or a salt, hydrate, deuterated analog, or fluorinated analog thereof, wherein
Ar is: 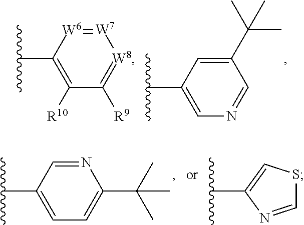
W¹ is chosen from N—R¹, O, and S, or when W⁹ is N, W¹ may additionally be C—R⁵⁰;
W² is C—R² or N;
W³ is C—R³ or N;
W⁴ is C—R⁴ or N;
W⁵ is C—R⁵ or N;
W⁶ is C—R⁶ or N;
W⁷ is C—R⁷ or N;
W⁸ is C—R⁸ or N;
W⁹ is C, or when W¹ is C—R⁵⁰, W⁹ may be N;
R¹ is selected from H, (C₁-C₃)alkyl, —CH₂OC(═O)R³⁰, —CH₂OP(═O)OR⁴⁰OR⁴¹, —C(═O)OR⁴², and —C(═O)R⁴³;
R², R³, R⁴, and R⁵ are selected independently from hydrogen, deuterium, halogen, perfluoro(C₁-C₄)alkyl, (C₁-C₄)alkyl, (C₁-C₄)alkoxy, perfluoro(C₁-C₄)alkoxy, (C₁-C₄)acyl, (C₁-C₄)alkoxy(C₁-C₄)alkyl, hydroxy(C₁-C₄)alkyl, hydroxy, carboxy, (C₁-C₄)alkoxycarbonylamino, carboxamido, (C₁-C₄)alkylaminocarbonyl, cyano, acetoxy, nitro, amino, (C₁-C₄)alkylamino, di(C₁-C₄)alkylamino, mercapto, (C₁-C₄)alkylthio, aminosulfonyl, (C₁-C₄)alkylsulfonyl, and (C₁-C₄)acylamino;
R⁶ and R¹⁰ are selected independently from hydrogen, deuterium, halo, (C₁-C₃)alkyl, perfluoro(C₁-C₃)alkyl, hydroxy, (C₁-C₃)alkoxy, perfluoro(C₁-C₃)alkoxy, and amino;
R⁷ and R⁹ are selected independently from hydrogen, deuterium, hydroxy, cyano, amino, halogen, halo(C₁-C₄)alkyl, (C₁-C₄)alkoxy, halo(C₁-C₄)alkoxy, 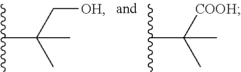
R⁸ is selected from hydrogen, deuterium, halogen, halo(C₁-C₄)alkyl, (C₁-C₄)alkyl, (C₁-C₄)alkoxy, halo(C₁-C₄)alkoxy, cyano, phenyl, phenoxy, benzyloxy, amino, 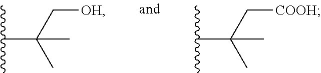
R³⁰ is selected from (C₁-C₁₀)hydrocarbyl, (C₁-C₁₀)hydrocarbyl substituted with amino, (C₁-C₁₀)hydrocarbyl substituted with (C₁-C₄)hydrocarbyl, (C₁-C₁₀)hydrocarbyl substituted with carboxyl, carboxy, (C₁-C₆)alkoxycarbonyl, (C₁-C₆)alkoxycarbonylamino, methylthio, heterocyclyl, (C₁-C₁₀)oxaalkyl, CHR⁴⁴NHR⁴⁵ and guanidine;
R⁴⁰ and R⁴¹ are selected independently from hydrogen (C₁-C₆)hydrocarbyl;
R⁴² is (C₁-C₅)alkyl;
R⁴³ is (C₁-C₃)alkyl,
R⁴⁴ is selected from any naturally occurring amino acid sidechain;
R⁴⁵ is selected from H, methyl, and (C₁-C₄)alkoxycarbonyl; and
R⁵⁰ is H or (C₁-C₃)alkyl.

33-53. (canceled)

54. The method of claim 1, wherein the compound is selected from the Compound I 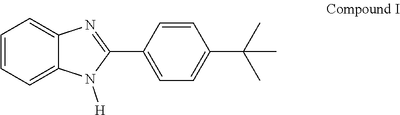 [SMILES: CC(C)(C)c1ccc(-c2nc3ccccc3[nH]2)cc1]
and Compound II 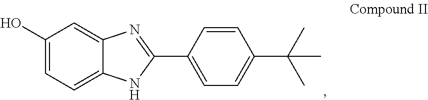 [SMILES: CC(C)(C)c1ccc(-c2nc3cc(O)ccc3[nH]2)cc1]
or a pharmaceutically acceptable salt, solvate, hydrate, or deuterated analog thereof.

55. The method of claim 54, wherein the compound is Compound I or a pharmaceutically acceptable salt, hydrate, or deuterated analog thereof.

56. The method of claim 54, wherein the compound is Compound II or a pharmaceutically acceptable salt, hydrate, or deuterated analog thereof.

57. The method of claim 4, wherein the compound or salt, solvate, hydrate, deuterated analog, or fluorinated analog thereof is administered in a suspension, a solution, or in a solid dosage form.

58. The method of claim 57, wherein the solid dosage form is a capsule.

59. The method of claim 57, wherein the solid dosage form is a tablet.
